SEQUENCE LISTING (3 sequences)

SEQ ID NO: 1 - protein, 11 aa, Homo sapiens
  REGION 1..11: note = Description of sequence: Carbamidomethylated peptide
SLAPQAGTEPC

SEQ ID NO: 2 - protein, 7 aa, Homo sapiens
  REGION 1..7: note = Description of sequence: Carbamidomethylated peptide
PPEAAAC

SEQ ID NO: 3 - protein, 12 aa, Homo sapiens
LLGGSKPVNLGR

(19) United States
(12) Patent Application Publication
(10) Pub. No.: US 2026/0338782 A1
(43) Pub. Date: Oct. 29, 2026

(54) METHODS AND COMPOSITIONS FOR DETECTING CANCER USING EXOSOMAL PROTEINS

(71) Applicant: Ohio State Innovation Foundation, Columbus, OH (US)

(72) Inventor: Selvendiran KARUPPAIYAH, Columbus, OH (US)

(21) Appl. No.: 19/107,790

(22) PCT Filed: Sep. 1, 2023
(86) PCT No.: PCT/US2023/073325

§ 371 Date: Feb. 28, 2025

Related U.S. Application Data
(60) Provisional application No. 63/403,452, filed on Sep. 2, 2022.
(60) Provisional application No. 63/406,481, filed on Sep. 14, 2022.

(51) Int. Cl. G01N 33/57545

(52) U.S. Cl. CPC G01N 33/57545

(57) ABSTRACT

The present disclosure relates to the use of exosomal proteins to identify, diagnose, treat, and/or prevent ovarian cancer.

### CROSS REFERENCE TO RELATED APPLICATIONS

This application claims priority to, and the benefit of, U.S. Provisional Patent Application No. 63/403,452, filed Sep. 2, 2022, and U.S. Provisional Patent Application No. 63/406,481, filed Sep. 14, 2022, each of which is entitled “METHODS AND COMPOSITIONS FOR DETECTING CANCER USING EXOSOMAL PROTEINS,” and each of which is incorporated by reference herein in its entirety.

### STATEMENT REGARDING FEDERALLY SPONSORED RESEARCH

This invention was made with government support under W81XWH-22-1-0371 and W81XWH-21-1-0427 awarded by the Army Medical Research and Development Command. The government has certain rights in the invention.

### REFERENCE TO SEQUENCE LISTING

The sequence listing submitted on Sep. 1, 2023, as an XML file entitled “103361-357WO1_ST26.xml” created on Aug. 28, 2023, and having a file size of 4,384 bytes is hereby incorporated by reference pursuant to 37 C.F.R. § 1.52 (e) (5).

### FIELD

The present disclosure relates to the use of exosomal proteins to identify, diagnose, treat, and/or prevent ovarian cancer.

### BACKGROUND

Ovarian cancer is the deadliest of all gynecologic cancers. High-grade serous ovarian carcinoma (HGSOC), the most common histologic subtype, is an aggressive disease with a poor prognosis. This owes largely to the fact that most patients with HGSOC are diagnosed with advanced-stage disease, and there is a significant difference in survival between early- and advanced-stage disease: the 5-year survival rate for patients diagnosed with stage 1 or stage 2 HGSOC is 66.9-81.3% compared to 33.3-41.3% for patients diagnosed at stage 3 or 4. Despite its high variability in expression and lack of specificity for early-stage disease, CA-125 remains the most commonly used biomarker for HGSOC. One limitation to successfully identifying early detection biomarkers is the fact that most are studied in whole serum while many cancer biomarkers are serum proteins found in low abundance, making them difficult to reliably detect with existing methods.

Exosomes are a unique source of biomarkers as they can be obtained from any bodily fluid, are highly stable, and reflect the information of their cell of origin. Exosomes are also capable of reprograming normal cells to more malignant phenotype and recognized as mediators of disease progression and metastasis. Moreover, exosome isolation and identification is useful for clinical translation. Using pre-treatment samples from patients with HGSOC, exosomal proteins that are differentially expressed in women with early-stage HGSOC compared to controls and advanced-stage HGSOC were identified. Herein, it is contemplated that unique exosomal protein signatures can serve as sensitive and specific early-detection biomarkers for the early detection of HGSOC.

Given limitations of early detection of cancer biomarkers, there is need to address the aforementioned problems mentioned above by identifying biomarkers located in exosomes to identify, diagnosis, treat, and/or prevent ovarian cancer, including, but not limited to HGSOC. The methods disclosed herein address these and other needs.

### SUMMARY

The present disclosure provides methods of identifying, diagnosing, staging, tracking, treating, and/or preventing an ovarian cancer, such as for example a high grade serous ovarian carcinoma (HGSOC).

In one aspect, disclosed herein is a method of diagnosing a subject with a high grade serous ovarian carcinoma (HGSOC), the method comprising measuring at least one exosomal proteins selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, phosphorylated STAT3 (pSTAT3), TETN, TETR, or VEGF1, wherein the subject comprises an increased amount of the one or more exosomal proteins relative to a healthy control subject, and wherein the increased amount of the one or more exosomal proteins identifies an ovarian cancer.

In one aspect, disclosed herein is a method of treating or preventing a high grade serous ovarian carcinoma (HGSOC) in a subject, the method comprising isolating a serum sample from the subject, measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, or VEGF1 in the serum sample, detecting the HGSOC in the subject comprising increased levels of the at least one exosomal protein relative to a control subject, and administering to the subject an anti-cancer therapy selected from chemotherapy, immunotherapy, radiation therapy, surgery, palliative care, or combinations thereof.

In one aspect disclosed herein is a method of identifying a stage of ovarian cancer in a subject, the method comprising isolating a serum sample from the subject, measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, or VEGF1 in the tissue sample, and identifying an early stage ovarian cancer in the tissue sample comprising increased expression of CFH, CCNE1, FAS, IL-6, PZP, (p)STAT3, or combinations thereof compared to a healthy control, identifying the early stage ovarian cancer in the tissue sample comprising a differential expression of CFH, CCNE1, IL-6, PZP, (p)STAT3, VEGF, or combinations thereof compared to a subject with an advanced stage ovarian cancer, or identifying an advanced stage of ovarian cancer in the tissue sample comprising an increased expression of CFH, IL-6, PZP, (p)STAT3, VEGF, or combinations thereof compared to the healthy control.

In one aspect, disclosed herein is a method of tracking progression of an ovarian cancer in a subject, the method comprising isolating at least two serum samples from the subject, measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, or VEGF1 in the at least two serum samples, and identifying a progression from a healthy ovary to a cancerous ovary when a second serum sample comprises altered levels of ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, VEGF1, or combinations thereof compared to a first serum sample.

In some embodiments, two, three, four, five or more serum samples are isolated from the subject. In some embodiments, the cancerous ovary comprises an early stage ovarian cancer or an advanced stage ovarian cancer. In some embodiments, the cancer ovary comprises a high grade serous ovarian carcinoma (HGSOC).

In some embodiments, the early stage ovarian cancer comprises increased expression of CFH, CCNE1, FAS, IL-6, PZP, (p)STAT3, or combinations thereof relative to the healthy ovary. In some embodiments, the advanced stage ovarian cancer comprises increased expression of CFH, IL-6, PZP, (p)STAT3, VEGF, or combinations thereof relative to the healthy ovary. In some embodiments, the advanced stage ovarian cancer comprises altered expression of CFH, CCNE1, IL-6, PZP, STAT3, VEGF, or combinations thereof relative to the early stage ovarian cancer.

In some embodiments, the method measures at least 1 exosomal protein to about 30 exosomal proteins. In some embodiments, the at least one exosomal proteins are measured in a serum sample.

In some embodiments, the method distinguishes between an early stage HGSOC and an advanced stage HGSOC. In some embodiments, the exosomal proteins are increased in a subject with the early stage HGSOC subject relative to the healthy control subject. In some embodiments, the exosomal proteins are increased in a subject with the advanced stage HGSOC subject relative to the healthy control subject. In some embodiments, the exosomal proteins are differentially expressed in the subject with the early stage HGSOC subject relative to the subject with the advanced stage HGSOC.

In some embodiments, the method further detects a protein not located in an exosome. In some embodiments, the method further detects HE4, MUC16 (CA-125), or combination thereof.

In some embodiments, the at least one exosomal proteins are measured using a protein detection technique selected from liquid chromatography, mass spectrometry, trapped ion mobility spectrometry (TIMS), time-of-flight (TOF) mass spectrometry, enzyme linked immunoassay (ELISA), or combinations thereof.

In some embodiments, the subject diagnosed with the ovarian cancer is administered an anti-cancer therapy. In some embodiments, the method comprises administering an anti-cancer therapy to the subject with an ovarian cancer. In some embodiments, the anti-cancer therapy comprises chemotherapy, immunotherapy, radiation therapy, surgery, palliative care, or combinations thereof.

In some embodiments, the ovarian cancer comprises a high grade serous ovarian carcinoma (HGSOC).

### BRIEF DESCRIPTION OF FIGURES

The accompanying figures, which are incorporated in and constitute a part of this specification, illustrate several aspects described below.

FIGS. 1A and 1B show the schematic of discovery phase and confirmation of exosomal proteins in HGSOC. Workflow of the serum exosome proteome analysis for discovery and confirmation of differentially expressed exosomal proteins candidates in three groups. FIG. 1A shows the serum exosomal proteins isolated by ultracentrifugation or exoquick kit or MED from controls, early- and advanced-stage HGSOC samples were subjected to LC-MS/MS or shot-gun proteomics. Exosomal proteins were chosen for confirmation based on fold change and statistical significance. FIG. 1B shows the confirmation of candidate proteins using ELISA, PEA, PRM, Luminex and Mass Spectrometry in new set of patient samples from PI or collaborator institutions.

FIGS. 2A, 2B, 2C, 2D, 2E and 2F show the exosome isolation and confirmation. FIG. 2Ai shows the image of Microfluidic device (MFD), MED channels functionalized with CD9, CD63 and TSG101 antibodies, the flow is shown in different colors. FIG. 2Aii shows the exo-FITC labelled Exosome (green) capture in MFD channels. FIGS. 2Aii and 2Aiv show the morphological characterization and size measurement of exosomes (indicated by red arrows) by (FIG. 2Aiii) Cryo and (FIG. 2Aiv) classical transmission electron microscope (TEM). FIGS. 2B and 2C show the confirmation of exosome specific marker CD9 and CD63 population by flowcytometry in the different patient' serum samples. FIG. 2D shows the exosome particles confirmed by image stream flow cytometry (the bar surrounding color indicated exosome count) in control, benign and HGSOC serum stage I/II and stage IV samples. FIGS. 2E and 2F show the nanoparticle tracking analysis (NTA) for exosome quantification and size confirmation, isolated by microfluidic device (n=5).

FIGS. 3A, 3B, and 3C show the cumulative receiver operating characteristic (ROC) curves of exosomal protein expression measured by ELISA in the confirmation set of samples from OSU/Inova. FIGS. 3A and 3B show the ROC curves based on ELISA results from exosomes isolated from controls and early-stage HGSOC samples. FIG. 3C shows the exosomal protein expression analyzed by ELISA in a new set of early- and advanced-stage HGSOC samples.

FIGS. 4A, 4B, and 4C show the validation of candidate exosomal protein in new set of HGSOC and control serum samples by ELISA and Luminex. FIG. 4A shows the dot plot of differential expression of exosomal proteins as measured by ELISA in new set of patient samples. FIG. 4B shows the ROC curve of validation cohort with known biomarker of HE4: ROC curve analysis of identified exosomal protein candidate in the two validation cohorts combined compare with known biomarker of HE4. FIG. 4C shows the luminex assay results of candidate proteins available in Luminex panel.

FIGS. 5A, 5B, 5C, 5D, 5E, and 5F show the principal component analysis (PCA) and violin box for exosome samples. FIG. 5A shows the PCA for exosome samples after normalizing data and imputing missing values demonstrate clustering within groups and clear separation across groups. FIG. 5B shows the violin box for samples after normalization and imputing missing values. FIG. 5C shows the distribution overlay of missing values in exosome samples. Data in red is after filtering lowly expressed proteins and in blue follows imputation. FIG. 5D shows the scatter plot of matrices for exosome samples. SPLOM depicts the correlation and similarity between and across all samples and in total abundance. FIG. 5E shows the volcano plot depicting the protein profile of Control and early-stage HGSC exosome samples. The black dashed line is a significance threshold (q-value <0.05) while the blue dashed lines are a fold-change < or >2 (log FC>1, log FC<1). Proteins Down-regulated in patient exosomes are shown in red, those Upregulated in are in green. FIG. 5F shows the spectral count quantification analyzed by statistical method (n=4).

FIGS. 6A, 6B, and 6C show total exosomes and vesicle sub-population analysis in patient serum exosomes. FIG. 6A shows the comparison of the Total exosome particle count measured by Imagestream flow cytometry, shows the increased exosome counts correlating with the disease stage (n=9). FIG. 6B shows the exosome particle count (green) measured by Image stream flow cytometer to show the technical reproducibility of different MED channels (Ch-1 or Ch-2) using same patient sample. FIG. 6C shows the comparative Nanoparticle tracking analysis (NTA) for total EpCAM positive exosome tested in two different labs (PI and Co—I) with our MED channels shows good reproducibility and reliability.

FIGS. 7A, 7B, and 7C show the exosome isolation by exoquick kit and morphological characterization. FIGS. 7A and 7B show the exosome particle concentration and size confirmed by nanoparticle tracking analysis (NTA) in control, & HGSOC serum stage I/II and stage IV samples. FIG. 7C show the exosome Size measurement (indicated by red arrows) by classical transmission electron microscope (TEM).

FIGS. 8A, 8B, 8C, 8D, and 8E show the exosome isolation using MFD. FIG. 8A shows the image of microfluidic device (MFD). FIG. 8B shows the morphological characterization of MFD captured exosomes on the PDMS surface and exosome size measurement by SEM (200 nm scale bar. FIG. 8C shows the confirmation of exosome specific marker CD9, CD63 by dot blot and TSG101 by Western blot in the different patient' serum samples. FIGS. 8D and 8E show the comparison of the exosome subpopulation based on the positivity of exosome surface markers (CD9, CD63 and EpCAM) in Late stage HGSOC samples (n=9) and their respective unstained controls.

FIGS. 9A and 9B show the IPA core analysis on the top canonical pathways and diseases, and functions in the data set. FIG. 9A shows the top canonical pathways and the percentage of upregulated and downregulated proteins identified in our dataset expressed as log (P value) in Early HGSOC stage (I&II) Vs Control healthy patient samples. FIG. 9B shows the top disease and functions associated with the proteins identified in our dataset are expressed as log (P value) above a significance threshold based on Fisher exact test (P<0.05).

FIG. 10 shows the Protein expression in control, early & late stage HGSOC. Expression of CPN1, MMP3 and Pim-2 Oncogene expression in early stage HGSOC compare with control or late stage HGSOC tissues by shotgun proteomics or LC-MS/MS (n=9, p<0.05).

FIG. 11 shows The Cancer Genome Atlas (TCGA) Research Network with a slight difference in the percent (%) levels.

FIG. 12 shows the exosome protein expression in HGSOC patient serum samples. Based on ELISA data, heat map showing the differential expression of serum exosome proteins in control, benign, early- and advanced-stage HGSOC samples (n=6, p<0.05).

FIG. 13 shows the exosome size measurement (indicated by red arrows) by classical transmission electron microscope (TEM) in new set of control and early stage HGSOC serum sample.

FIG. 14 shows the image stream flow cytometry measurements on the total exosome particle count in controls vs early stage HGSOC samples (n=6).

FIGS. 15A and 15B show the Hierarchical Clustering analysis performed on TIMS-TOF exosome samples (Control and early stage HGSC) n=6 based on different methods (tims intensity and tims spec count) show that 6 of them are potential outliers out of the 12 samples and after excluding those samples the analysis show that control samples C2, C3, C6 and Early stage samples ES2, ES3, ES5 clustered well.

FIGS. 16A, 16B, 16C, and 16D show the Principal component analysis and violin box plot. FIGS. 16A and 16C show the PCA for exosome samples pre-(FIG. 16A) and post-filtering (FIG. 16C) demonstrate clustering within groups and clear separation across groups. FIGS. 16B and 16D show the violin box for samples pre-(FIG. 16B) and post-filtering (FIG. 16D).

FIGS. 17A, 17B, and 17C show the distribution of missing values in exosome samples during data processing. FIG. 17A shows the data before filtering lowly expressed protein, FIG. 17B shows the data after filtering, and FIG. 17C shows the data following imputation.

FIGS. 18A and 18B show the Parallel Reaction Monitoring. FIG. 18A shows the selected ion chromatograph of m/z=1106.8427 (3+) in a DDA run, as shown in the inserted spectrum, 1106.8247 (3+) coeluted with another peak at the same retention time; no MS/MS was detected for this peptide. FIG. 18B shows the base peaks of 1106.8247 (3+) in a targeted method. Signaling average at retention time ~40 min generated inserted MS/MS spectrum, which was identified as SLAPQAGTEPC (SEQ ID NO: 1) (Carbamidomethyl), PPEAAAC (SEQ ID NO: 2) (Carbamidomethyl), and LLGGSKPVNLGR (SEQ ID NO: 3) from human Cation-independent mannose-6-phosphate receptor [MPRI_HUMAN].

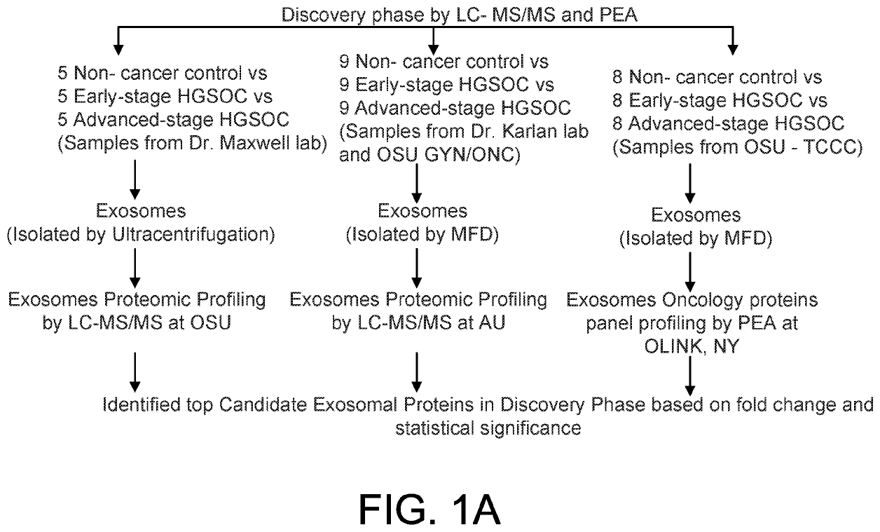

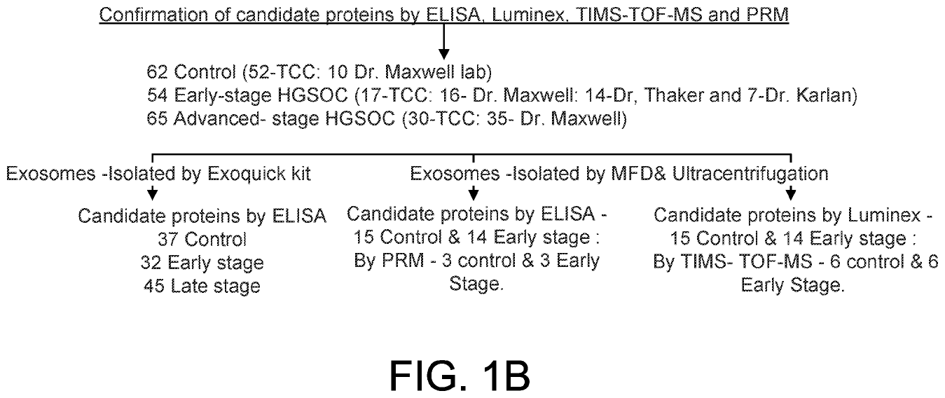

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### TERMINOLOGY

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The terms “exosomes”, “micro-vesicles” and “extracellular vesicles” are herein used interchangeably. Exosomes are membrane-bound extracellular vesicles that are produced in the endosomal compartment of most eukaryotic cells. The multivesicular body (MVB) is an endosome with intraluminal vesicles (ILV) that bud inward into the endosomal lumen. If the MVB fuses with the cell surface, these ILVs are released as exosomes.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, the term “detect”, “detected” or “detecting” includes measure, measured or measuring.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

A “control” is an alternative subject or sample used in an experiment for comparison purposes. A control can be “positive” or “negative.”

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit means any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide.

The terms “anticancer” and “anticancer therapy” refers to a substance, composition, or formula that counteracts the effects or inhibits the development of a cancerous cells and tissues. Said therapies also refer to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, “tracking” refers to the process of identifying, assessing, and/or managing subjects who have been diagnosed with a disease or disorder (such as, for example a cancer), and comparing the subject's current disease state or symptoms to their prior disease state or symptoms to determine whether said disease or disorder is improving or worsening.

As used herein, “early stage” refers to a timepoint immediately after onset of a disease or disorder (such as, for example cancer), wherein symptoms can be mild, nonspecific, or asymptomatic. This stage can range from hours for some diseases, to days, weeks, or years for other diseases.

As used herein, the terms “advanced staged” or “late stage” refers to a timepoint after onset of a disease or disorder, wherein symptoms include, but are not limited to moderate, severe, specific, acute, chronic, and combinations thereof. Specifically, advanced stage cancers refer to a cancer or tumor that has continued growing even to where it has spread to additional organs aside from the original (primary) site, or a cancer or tumor that has returned (recurred) after initially being removed.

Methods of Identifying, Diagnosing, and/or Tracking an Ovarian Cancer

The present disclosure provides methods of identifying, diagnosing, staging, tracking, treating, and/or preventing an ovarian cancer, such as for example a high grade serous ovarian carcinoma (HGSOC). The present disclosure also provides methods of predicting the likelihood of ovarian cancer in a subject based on detecting or measuring the levels in a specimen or biological sample from the subject of the foregoing biomarkers. As described in this specification, a change in the expression levels of these biomarkers, particularly their relative expression levels, as compared with a control group of patients who do not have ovarian cancer, is predictive of ovarian cancer in that subject.

In one aspect, disclosed herein is a method of diagnosing a subject with a high grade serous ovarian carcinoma (HGSOC), the method comprising measuring at least one exosomal proteins selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, phosphorylated STAT3 (pSTAT3), TETN, TETR, or VEGF1, wherein the subject comprises an increased amount of the one or more exosomal proteins relative to a healthy control subject, and wherein the increased amount of the one or more exosomal proteins identifies an ovarian cancer.

In one aspect disclosed herein is a method of identifying a stage of ovarian cancer in a subject, the method comprising isolating a serum sample from the subject, measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, or VEGF1 in the tissue sample, and identifying an early stage ovarian cancer in the tissue sample comprising increased expression of CFH, CCNE1, FAS, IL-6, PZP, (p)STAT3, or combinations thereof compared to a healthy control, identifying the early stage ovarian cancer in the tissue sample comprising a differential expression of CFH, CCNE1, IL-6, PZP, (p)STAT3, VEGF, or combinations thereof compared to a subject with an advanced stage ovarian cancer, or identifying an advanced stage of ovarian cancer in the tissue sample comprising an increased expression of CFH, IL-6, PZP, (p)STAT3, VEGF, or combinations thereof compared to the healthy control.

In one aspect, disclosed herein is a method of tracking progression of an ovarian cancer in a subject, the method comprising isolating at least two serum samples from the subject, measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, or VEGF1 in the at least two serum samples, and identifying a progression from a healthy ovary to a cancerous ovary when a second serum sample comprises altered levels of ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, VEGF1, or combinations thereof compared to a first serum sample.

In one aspect, disclosed herein is a method of treating or preventing a high grade serous ovarian carcinoma (HGSOC) in a subject, the method comprising isolating a serum sample from the subject, measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, or VEGF1 in the serum sample, detecting the HGSOC in the subject comprising increased levels of the at least one exosomal protein relative to a control subject, and administering to the subject an anti-cancer therapy selected from chemotherapy, immunotherapy, radiation therapy, surgery, palliative care, or combinations thereof.

In some embodiments, one, two, three, four, five or more serum samples are isolated from the subject. In some embodiments, the cancerous ovary comprises an early stage ovarian cancer or an advanced stage ovarian cancer. In some embodiments, the cancer ovary comprises a high grade serous ovarian carcinoma (HGSOC).

In some embodiments, the early stage ovarian cancer comprises increased expression of CFH, CCNE1, FAS, IL-6, PZP, (p)STAT3, or combinations thereof relative to the healthy ovary. In some embodiments, the early stage ovarian cancer comprises increased expression of CCNE1 and CFH relative to the healthy ovary. In some embodiments, the early stage ovarian cancer comprises increased expression of CCNE1 and FAS relative to the healthy ovary. In some embodiments, the early stage ovarian cancer comprises increased expression of CCNE1 and IL-6 relative to the healthy ovary. In some embodiments, the early stage ovarian cancer comprises increased expression of CCNE1 and PZP relative to the healthy ovary. In some embodiments, the early stage ovarian cancer comprises increased expression of CCNE1 and STAT3 relative to the healthy ovary. In some embodiments, the early stage ovarian cancer comprises increased expression of CCNE1 and pSTAT3 relative to the healthy ovary.

In some embodiments, the early stage ovarian cancer comprises increased expression of CFH and FAS relative to the healthy ovary. In some embodiments, the early stage ovarian cancer comprises increased expression of CFH and IL-6 relative to the healthy ovary. In some embodiments, the early stage ovarian cancer comprises increased expression of CFH and PZP relative to the healthy ovary. In some embodiments, the early stage ovarian cancer comprises increased expression of CFH and STAT3 relative to the healthy ovary. In some embodiments, the early stage ovarian cancer comprises increased expression of CFH and pSTAT3 relative to the healthy ovary.

In some embodiments, the early stage ovarian cancer comprises increased expression of FAS and IL-6 relative to the healthy ovary. In some embodiments, the early stage ovarian cancer comprises increased expression of FAS and PZP relative to the healthy ovary. In some embodiments, the early stage ovarian cancer comprises increased expression of FAS and STAT3 relative to the healthy ovary. In some embodiments, the early stage ovarian cancer comprises increased expression of FAS and pSTAT3 relative to the healthy ovary.

In some embodiments, the early stage ovarian cancer comprises increased expression of IL-6 and PZP relative to the healthy ovary. In some embodiments, the early stage ovarian cancer comprises increased expression of IL-6 and STAT3 relative to the healthy ovary. In some embodiments, the early stage ovarian cancer comprises increased expression of IL-6 and pSTAT3 relative to the healthy ovary.

In some embodiments, the early stage ovarian cancer comprises increased expression of pSTAT3 and STAT3 relative to the healthy ovary. In some embodiments, the early stage ovarian cancer comprises increased expression of pSTAT3 and PZP relative to the healthy ovary.

In some embodiments, the early stage ovarian cancer comprises increased expression of STAT3 and PZP relative to the healthy ovary.

In some embodiments, the early stage ovarian cancer comprises increased expression of CCNE1, CFH, and FAS relative to the healthy ovary. In some embodiments, the early stage ovarian cancer comprises increased expression of CCNE1, CFH, and IL-6 relative to the healthy ovary. In some embodiments, the early stage ovarian cancer comprises increased expression of CCNE1, CFH, and pSTAT3 relative to the healthy ovary. In some embodiments, the early stage ovarian cancer comprises increased expression of CCNE1, CFH, and STAT3 relative to the healthy ovary. In some embodiments, the early stage ovarian cancer comprises increased expression of CCNE1, CFH, and pSTAT3 relative to the healthy ovary. In some embodiments, the early stage ovarian cancer comprises increased expression of CCNE1, FAS, and IL-6 relative to the healthy ovary. In some embodiments, the early stage ovarian cancer comprises increased expression of CCNE1, FAS, and pSTAT3 relative to the healthy ovary. In some embodiments, the early stage ovarian cancer comprises increased expression of CCNE1, FAS, and STAT3 relative to the healthy ovary. In some embodiments, the early stage ovarian cancer comprises increased expression of CCNE1, FAS, and PZP relative to the healthy ovary.

In some embodiments, the early stage ovarian cancer comprises increased expression of CCNE1, IL-6, and pSTAT3 relative to the healthy ovary. In some embodiments, the early stage ovarian cancer comprises increased expression of CCNE1, IL-6, and STAT3 relative to the healthy ovary. In some embodiments, the early stage ovarian cancer comprises increased expression of CCNE1, IL-6, and PZP relative to the healthy ovary. In some embodiments, the early stage ovarian cancer comprises increased expression of CCNE1, STAT3, and pSTAT3 relative to the healthy ovary. In some embodiments, the early stage ovarian cancer comprises increased expression of CCNE1, PZP, and pSTAT3 relative to the healthy ovary. In some embodiments, the early stage ovarian cancer comprises increased expression of CCNE1, PZP, and STAT3 relative to the healthy ovary.

In some embodiments, the early stage ovarian cancer comprises increased expression of CFH, FAS, and IL-6 relative to the healthy ovary. In some embodiments, the early stage ovarian cancer comprises increased expression of CFH, FAS, and PZP relative to the healthy ovary. In some embodiments, the early stage ovarian cancer comprises increased expression of CFH, FAS, and STAT3 relative to the healthy ovary. In some embodiments, the early stage ovarian cancer comprises increased expression of CFH, FAS, and pSTAT3 relative to the healthy ovary.

In some embodiments, the early stage ovarian cancer comprises increased expression of CFH, IL-6, and PZP relative to the healthy ovary. In some embodiments, the early stage ovarian cancer comprises increased expression of CFH, IL-6, and STAT3 relative to the healthy ovary. In some embodiments, the early stage ovarian cancer comprises increased expression of CFH, IL-6, and pSTAT3 relative to the healthy ovary. In some embodiments, the early stage ovarian cancer comprises increased expression of CFH, PZP, and STAT3 relative to the healthy ovary. In some embodiments, the early stage ovarian cancer comprises increased expression of CFH, PZP, and pSTAT3 relative to the healthy ovary. In some embodiments, the early stage ovarian cancer comprises increased expression of CFH, pSTAT3, and STAT3 relative to the healthy ovary.

In some embodiments, the early stage ovarian cancer comprises increased expression of CCNE1, FAS, and IL-6 relative to the healthy ovary. In some embodiments, the early stage ovarian cancer comprises increased expression of CCNE1, FAS, and PZP relative to the healthy ovary. In some embodiments, the early stage ovarian cancer comprises increased expression of CCNE1, FAS, and STAT3 relative to the healthy ovary. In some embodiments, the early stage ovarian cancer comprises increased expression of CCNE1, FAS, and pSTAT3 relative to the healthy ovary. In some embodiments, the early stage ovarian cancer comprises increased expression of CCNE1, IL-6, and PZP relative to the healthy ovary. In some embodiments, the early stage ovarian cancer comprises increased expression of CCNE1, IL-6, and STAT3 relative to the healthy ovary. In some embodiments, the early stage ovarian cancer comprises increased expression of CCNE1, IL-6, and pSTAT3 relative to the healthy ovary. In some embodiments, the early stage ovarian cancer comprises increased expression of CCNE1, PZP, and STAT3 relative to the healthy ovary. In some embodiments, the early stage ovarian cancer comprises increased expression of CCNE1, PZP, and pSTAT3 relative to the healthy ovary. In some embodiments, the early stage ovarian cancer comprises increased expression of CCNE1, pSTAT3, and STAT3 relative to the healthy ovary.

In some embodiments, the early stage ovarian cancer comprises increased expression of FAS, IL-6, and PZP relative to the healthy ovary. In some embodiments, the early stage ovarian cancer comprises increased expression of FAS, IL-6, and STAT3 relative to the healthy ovary. In some embodiments, the early stage ovarian cancer comprises increased expression of FAS, IL-6, and pSTAT3 relative to the healthy ovary. In some embodiments, the early stage ovarian cancer comprises increased expression of FAS, PZP, and STAT3 relative to the healthy ovary. In some embodiments, the early stage ovarian cancer comprises increased expression of FAS, PZP, and pSTAT3 relative to the healthy ovary. In some embodiments, the early stage ovarian cancer comprises increased expression of FAS, pSTAT3, and STAT3 relative to the healthy ovary.

In some embodiments, the early stage ovarian cancer comprises increased expression of IL-6, PZP, and STAT3 relative to the healthy ovary. In some embodiments, the early stage ovarian cancer comprises increased expression of IL-6, PZP, and pSTAT3 relative to the healthy ovary. In some embodiments, the early stage ovarian cancer comprises increased expression of IL-6, pSTAT3, and STAT3 relative to the healthy ovary. In some embodiments, the early stage ovarian cancer comprises increased expression of PZP, STAT3, and pSTAT3 relative to the healthy ovary.

In some embodiments, the early stage ovarian cancer comprises increased expression of CFH, CCNE1, FAS, and IL-6 relative to the healthy ovary. In some embodiments, the early stage ovarian cancer comprises increased expression of CFH, CCNE1, FAS, and PZP relative to the healthy ovary. In some embodiments, the early stage ovarian cancer comprises increased expression of CFH, CCNE1, FAS, and STAT3 relative to the healthy ovary. In some embodiments, the early stage ovarian cancer comprises increased expression of CFH, CCNE1, FAS, and pSTAT3 relative to the healthy ovary.

In some embodiments, the early stage ovarian cancer comprises increased expression of CFH, CCNE1, IL-6, and PZP relative to the healthy ovary. In some embodiments, the early stage ovarian cancer comprises increased expression of CFH, CCNE1, IL-6, and STAT3 relative to the healthy ovary. In some embodiments, the early stage ovarian cancer comprises increased expression of CFH, CCNE1, IL-6, and pSTAT3 relative to the healthy ovary. In some embodiments, the early stage ovarian cancer comprises increased expression of CFH, CCNE1, PZP, and STAT3 relative to the healthy ovary. In some embodiments, the early stage ovarian cancer comprises increased expression of CFH, CCNE1, PZP, and pSTAT3 relative to the healthy ovary. In some embodiments, the early stage ovarian cancer comprises increased expression of CFH, CCNE1, pSTAT3, and STAT3 relative to the healthy ovary.

In some embodiments, the early stage ovarian cancer comprises increased expression of CFH, FAS, IL-6, and PZP relative to the healthy ovary. In some embodiments, the early stage ovarian cancer comprises increased expression of CFH, FAS, IL-6, and STAT3 relative to the healthy ovary. In some embodiments, the early stage ovarian cancer comprises increased expression of CFH, FAS, IL-6, and pSTAT3 relative to the healthy ovary. In some embodiments, the early stage ovarian cancer comprises increased expression of CFH, FAS, PZP, and STAT3 relative to the healthy ovary. In some embodiments, the early stage ovarian cancer comprises increased expression of CFH, FAS, PZP, and pSTAT3 relative to the healthy ovary. In some embodiments, the early stage ovarian cancer comprises increased expression of CFH, FAS, pSTAT3, and STAT3 relative to the healthy ovary.

In some embodiments, the early stage ovarian cancer comprises increased expression of CFH, IL-6, PZP, and STAT3 relative to the healthy ovary. In some embodiments, the early stage ovarian cancer comprises increased expression of CFH, IL-6, PZP, and pSTAT3 relative to the healthy ovary. In some embodiments, the early stage ovarian cancer comprises increased expression of CFH, IL-6, pSTAT3, and STAT3 relative to the healthy ovary. In some embodiments, the early stage ovarian cancer comprises increased expression of CFH, PZP, pSTAT3, and STAT3 relative to the healthy ovary.

In some embodiments, the early stage ovarian cancer comprises increased expression of CCNE1, FAS, IL-6, and PZP relative to the healthy ovary. In some embodiments, the early stage ovarian cancer comprises increased expression of CCNE1, FAS, IL-6, and STAT3 relative to the healthy ovary. In some embodiments, the early stage ovarian cancer comprises increased expression of CCNE1, FAS, IL-6, and pSTAT3 relative to the healthy ovary. In some embodiments, the early stage ovarian cancer comprises increased expression of CCNE1, FAS, PZP, and STAT3 relative to the healthy ovary. In some embodiments, the early stage ovarian cancer comprises increased expression of CCNE1, FAS, PZP, and pSTAT3 relative to the healthy ovary. In some embodiments, the early stage ovarian cancer comprises increased expression of CCNE1, FAS, pSTAT3, and STAT3 relative to the healthy ovary. In some embodiments, the early stage ovarian cancer comprises increased expression of CCNE1, IL-6, PZP, and STAT3 relative to the healthy ovary. In some embodiments, the early stage ovarian cancer comprises increased expression of CCNE1, IL-6, PZP, and pSTAT3 relative to the healthy ovary. In some embodiments, the early stage ovarian cancer comprises increased expression of CCNE1, IL-6, pSTAT3, and STAT3 relative to the healthy ovary. In some embodiments, the early stage ovarian cancer comprises increased expression of CCNE1, PZP, pSTAT3 and STAT3 relative to the healthy ovary.

In some embodiments, the early stage ovarian cancer comprises increased expression of CFH, FAS, IL-6, and PZP relative to the healthy ovary. In some embodiments, the early stage ovarian cancer comprises increased expression of CFH, FAS, IL-6, and STAT3 relative to the healthy ovary. In some embodiments, the early stage ovarian cancer comprises increased expression of CFH, FAS, IL-6, and pSTAT3 relative to the healthy ovary. In some embodiments, the early stage ovarian cancer comprises increased expression of CFH, FAS, PZP, and STAT3 relative to the healthy ovary. In some embodiments, the early stage ovarian cancer comprises increased expression of CFH, FAS, PZP, and pSTAT3 relative to the healthy ovary. In some embodiments, the early stage ovarian cancer comprises increased expression of CFH, FAS, STAT3, and pSTAT3 relative to the healthy ovary. In some embodiments, the early stage ovarian cancer comprises increased expression of CFH, IL-6, PZP, and STAT3 relative to the healthy ovary. In some embodiments, the early stage ovarian cancer comprises increased expression of CFH, IL-6, PZP, and pSTAT3 relative to the healthy ovary. In some embodiments, the early stage ovarian cancer comprises increased expression of CFH, IL-6, pSTAT3, and STAT3 relative to the healthy ovary. In some embodiments, the early stage ovarian cancer comprises increased expression of CFH, PZP, pSTAT3, and STAT3 relative to the healthy ovary.

In some embodiments, the early stage ovarian cancer comprises increased expression of FAS, IL-6, PZP, and STAT3 relative to the healthy ovary. In some embodiments, the early stage ovarian cancer comprises increased expression of FAS, IL-6, PZP, and pSTAT3 relative to the healthy ovary. In some embodiments, the early stage ovarian cancer comprises increased expression of FAS, IL-6, pSTAT3, and STAT3 relative to the healthy ovary. In some embodiments, the early stage ovarian cancer comprises increased expression of FAS, PZP, pSTAT3, and STAT3 relative to the healthy ovary. In some embodiments, the early stage ovarian cancer comprises increased expression of IL-6, PZP, STAT3, and pSTAT3 relative to the healthy ovary.

In some embodiments, the early stage ovarian cancer comprises increased expression of CCNE1, CFH, FAS, IL-6, and PZP relative to the healthy ovary. In some embodiments, the early stage ovarian cancer comprises increased expression of CCNE1, CFH, FAS, IL-6, and STAT3 relative to the healthy ovary. In some embodiments, the early stage ovarian cancer comprises increased expression of CCNE1, CFH, FAS, IL-6, and pSTAT3 relative to the healthy ovary. In some embodiments, the early stage ovarian cancer comprises increased expression of CCNE1, CFH, FAS, PZP, and STAT3 relative to the healthy ovary. In some embodiments, the early stage ovarian cancer comprises increased expression of CCNE1, CFH, FAS, PZP, and pSTAT3 relative to the healthy ovary. In some embodiments, the early stage ovarian cancer comprises increased expression of CCNE1, CFH, FAS, pSTAT3, and STAT3 relative to the healthy ovary. In some embodiments, the early stage ovarian cancer comprises increased expression of CCNE1, CFH, IL-6, PZP, and STAT3 relative to the healthy ovary. In some embodiments, the early stage ovarian cancer comprises increased expression of CCNE1, CFH, IL-6, PZP, and pSTAT3 relative to the healthy ovary. In some embodiments, the early stage ovarian cancer comprises increased expression of CCNE1, CFH, IL-6, STAT3, and pSTAT3 relative to the healthy ovary. In some embodiments, the early stage ovarian cancer comprises increased expression of CCNE1, CFH, PZP, STAT3, and pSTAT3 relative to the healthy ovary.

In some embodiments, the early stage ovarian cancer comprises increased expression of CCNE1, FAS, IL-6, PZP, and STAT3 relative to the healthy ovary. In some embodiments, the early stage ovarian cancer comprises increased expression of CCNE1, FAS, IL-6, PZP, and pSTAT3 relative to the healthy ovary. In some embodiments, the early stage ovarian cancer comprises increased expression of CCNE1, FAS, IL-6, STAT3, and pSTAT3 relative to the healthy ovary. In some embodiments, the early stage ovarian cancer comprises increased expression of CCNE1, FAS, PZP, STAT3, and pSTAT3 relative to the healthy ovary. In some embodiments, the early stage ovarian cancer comprises increased expression of CCNE1, IL-6, PZP, STAT3, and pSTAT3 relative to the healthy ovary.

In some embodiments, the early stage ovarian cancer comprises increased expression of CFH, FAS, IL-6, PZP, and STAT3 relative to the healthy ovary. In some embodiments, the early stage ovarian cancer comprises increased expression of CFH, FAS, IL-6, PZP, and pSTAT3 relative to the healthy ovary. In some embodiments, the early stage ovarian cancer comprises increased expression of CFH, FAS, IL-6, STAT3, and pSTAT3 relative to the healthy ovary. In some embodiments, the early stage ovarian cancer comprises increased expression of CFH, FAS, PZP, STAT3, and pSTAT3 relative to the healthy ovary. In some embodiments, the early stage ovarian cancer comprises increased expression of CFH, IL-6, PZP, STAT3, and pSTAT3 relative to the healthy ovary. In some embodiments, the early stage ovarian cancer comprises increased expression of FAS, IL-6, PZP, STAT3, and pSTAT3 relative to the healthy ovary.

In some embodiments, the early stage ovarian cancer comprises increased expression of CCNE1, CFH, FAS, IL-6, PZP, and STAT3 relative to the healthy ovary. In some embodiments, the early stage ovarian cancer comprises increased expression of CCNE1, CFH, FAS, IL-6, PZP, and pSTAT3 relative to the healthy ovary. In some embodiments, the early stage ovarian cancer comprises increased expression of CCNE1, CFH, FAS, IL-6, pSTAT3, and STAT3 relative to the healthy ovary. In some embodiments, the early stage ovarian cancer comprises increased expression of CCNE1, CFH, FAS, PZP, pSTAT3, and STAT3 relative to the healthy ovary. In some embodiments, the early stage ovarian cancer comprises increased expression of CCNE1, CFH, IL-6, PZP, pSTAT3, and STAT3 relative to the healthy ovary. In some embodiments, the early stage ovarian cancer comprises increased expression of CCNE1, FAS, IL-6, PZP, pSTAT3, and STAT3 relative to the healthy ovary. In some embodiments, the early stage ovarian cancer comprises increased expression of CFH, FAS, IL-6, PZP, pSTAT3, and STAT3 relative to the healthy ovary.

In some embodiments, the early stage ovarian cancer comprises increased expression of CCNE1, CFH, FAS, IL-6, PZP, STAT3, and pSTAT3 relative to the healthy ovary.

In some embodiments, the advanced stage ovarian cancer comprises increased expression of CFH, IL-6, PZP, (p)STAT3, VEGF, or combinations thereof relative to the healthy ovary. In some embodiments, the advanced stage ovarian cancer comprises increased expression of pSTAT3 and CFH relative to the healthy ovary. In some embodiments, the advanced stage ovarian cancer comprises increased expression of pSTAT3 and IL-6 relative to the healthy ovary. In some embodiments, the advanced stage ovarian cancer comprises increased expression of pSTAT3 and PZP relative to the healthy ovary. In some embodiments, the advanced stage ovarian cancer comprises increased expression of pSTAT3 and STAT3 relative to the healthy ovary. In some embodiments, the advanced stage ovarian cancer comprises increased expression of pSTAT3 and VEGF relative to the healthy ovary. In some embodiments, the advanced stage ovarian cancer comprises increased expression of CFH and IL-6 relative to the healthy ovary. In some embodiments, the advanced stage ovarian cancer comprises increased expression of CFH and PZP relative to the healthy ovary. In some embodiments, the advanced stage ovarian cancer comprises increased expression of CFH and STAT3 relative to the healthy ovary. In some embodiments, the advanced stage ovarian cancer comprises increased expression of CFH and VEGF relative to the healthy ovary. In some embodiments, the advanced stage ovarian cancer comprises increased expression of IL-6 and PZP relative to the healthy ovary. In some embodiments, the advanced stage ovarian cancer comprises increased expression of IL-6 and STAT3 relative to the healthy ovary. In some embodiments, the advanced stage ovarian cancer comprises increased expression of IL-6 and VEGF relative to the healthy ovary. In some embodiments, the advanced stage ovarian cancer comprises increased expression of PZP and STAT3 relative to the healthy ovary. In some embodiments, the advanced stage ovarian cancer comprises increased expression of PZP and VEGF relative to the healthy ovary. In some embodiments, the advanced stage ovarian cancer comprises increased expression of STAT3 and VEGF relative to the healthy ovary.

In some embodiments, the advanced stage ovarian cancer comprises increased expression of pSTAT3, CFH, and IL-6 relative to the healthy ovary. In some embodiments, the advanced stage ovarian cancer comprises increased expression of pSTAT3, CFH, and PZP relative to the healthy ovary. In some embodiments, the advanced stage ovarian cancer comprises increased expression of pSTAT3, CFH, and STAT3 relative to the healthy ovary. In some embodiments, the advanced stage ovarian cancer comprises increased expression of pSTAT3, CFH, and VEGF relative to the healthy ovary. In some embodiments, the advanced stage ovarian cancer comprises increased expression of pSTAT3, IL-6, and PZP relative to the healthy ovary. In some embodiments, the advanced stage ovarian cancer comprises increased expression of pSTAT3, IL-6, and STAT3 relative to the healthy ovary. In some embodiments, the advanced stage ovarian cancer comprises increased expression of pSTAT3, IL-6, and VEGF relative to the healthy ovary. In some embodiments, the advanced stage ovarian cancer comprises increased expression of pSTAT3, PZP, and STAT3 relative to the healthy ovary. In some embodiments, the advanced stage ovarian cancer comprises increased expression of pSTAT3, PZP, and VEGF relative to the healthy ovary. In some embodiments, the advanced stage ovarian cancer comprises increased expression of pSTAT3, STAT, and VEGF relative to the healthy ovary. In some embodiments, the advanced stage ovarian cancer comprises increased expression of CFH, IL-6, and PZP relative to the healthy ovary. In some embodiments, the advanced stage ovarian cancer comprises increased expression of CFH, IL-6, and STAT3 relative to the healthy ovary. In some embodiments, the advanced stage ovarian cancer comprises increased expression of CFH, IL-6, and VEGF relative to the healthy ovary. In some embodiments, the advanced stage ovarian cancer comprises increased expression of CFH, PZP, and STAT3 relative to the healthy ovary. In some embodiments, the advanced stage ovarian cancer comprises increased expression of CFH, PZP, and VEGF relative to the healthy ovary. In some embodiments, the advanced stage ovarian cancer comprises increased expression of CFH, STAT3, and VEGF relative to the healthy ovary. In some embodiments, the advanced stage ovarian cancer comprises increased expression of IL-6, PZP, and STAT3 relative to the healthy ovary. In some embodiments, the advanced stage ovarian cancer comprises increased expression of IL-6, PZP, and VEGF relative to the healthy ovary. In some embodiments, the advanced stage ovarian cancer comprises increased expression of IL-6, STAT3, and VEGF relative to the healthy ovary. In some embodiments, the advanced stage ovarian cancer comprises increased expression of PZP, PZP, and STAT3 relative to the healthy ovary.

In some embodiments, the advanced stage ovarian cancer comprises increased expression of pSTAT3, CFH, IL-6, and PZP relative to the healthy ovary. In some embodiments, the advanced stage ovarian cancer comprises increased expression of pSTAT3, CFH, IL-6, and STAT3 relative to the healthy ovary. In some embodiments, the advanced stage ovarian cancer comprises increased expression of pSTAT3, CFH, IL-6, and VEGF relative to the healthy ovary. In some embodiments, the advanced stage ovarian cancer comprises increased expression of pSTAT3, CFH, PZP, and STAT3 relative to the healthy ovary. In some embodiments, the advanced stage ovarian cancer comprises increased expression of pSTAT3, CFH, PZP, and VEGF relative to the healthy ovary. In some embodiments, the advanced stage ovarian cancer comprises increased expression of pSTAT3, CFH, STAT3, and VEGF relative to the healthy ovary. In some embodiments, the advanced stage ovarian cancer comprises increased expression of pSTAT3, IL-6, PZP, and STAT3 relative to the healthy ovary. In some embodiments, the advanced stage ovarian cancer comprises increased expression of pSTAT3, IL-6, PZP, and VEGF relative to the healthy ovary. In some embodiments, the advanced stage ovarian cancer comprises increased expression of pSTAT3, IL-6, STAT3, and VEGF relative to the healthy ovary. In some embodiments, the advanced stage ovarian cancer comprises increased expression of pSTAT3, PZP, STAT3, and VEGF relative to the healthy ovary. In some embodiments, the advanced stage ovarian cancer comprises increased expression of CFH, IL-6, PZP, and STAT3 relative to the healthy ovary. In some embodiments, the advanced stage ovarian cancer comprises increased expression of CFH, IL-6, PZP, and VEGF relative to the healthy ovary. In some embodiments, the advanced stage ovarian cancer comprises increased expression of CFH, IL-6, STAT3, and VEGF relative to the healthy ovary. In some embodiments, the advanced stage ovarian cancer comprises increased expression of CFH, PZP, STAT3, and VEGF relative to the healthy ovary. In some embodiments, the advanced stage ovarian cancer comprises increased expression of IL-6, PZP, STAT3, and VEGF relative to the healthy ovary.

In some embodiments, the advanced stage ovarian cancer comprises increased expression of pSTAT3, CFH, IL-6, PZP, and STAT3 relative to the healthy ovary. In some embodiments, the advanced stage ovarian cancer comprises increased expression of pSTAT3, CFH, IL-6, PZP, and VEGF relative to the healthy ovary. In some embodiments, the advanced stage ovarian cancer comprises increased expression of pSTAT3, CFH, IL-6, STAT3, and VEGF relative to the healthy ovary. In some embodiments, the advanced stage ovarian cancer comprises increased expression of pSTAT3, CFH, PZP, STAT3, and VEGF relative to the healthy ovary. In some embodiments, the advanced stage ovarian cancer comprises increased expression of pSTAT3, IL-6, PZP, STAT3, and VEGF relative to the healthy ovary. In some embodiments, the advanced stage ovarian cancer comprises increased expression of CFH, IL-6, PZP, STAT3, and VEGF relative to the healthy ovary.

In some embodiments, the advanced stage ovarian cancer comprises increased expression of CFH, pSTAT3, IL-6, PZP, STAT3, and VEGF relative to the healthy ovary.

In some embodiments, the advanced stage ovarian cancer comprises altered expression of CFH, CCNE1, IL-6, PZP, STAT3, VEGF, or combinations thereof relative to the early stage ovarian cancer. In some embodiments, the advanced stage ovarian cancer comprises altered expression of CCNE1 and CFH relative to the early stage ovarian cancer. In some embodiments, the advanced stage ovarian cancer comprises altered expression of CCNE1 and IL-6 relative to the early stage ovarian cancer. In some embodiments, the advanced stage ovarian cancer comprises altered expression of CCNE1 and PZP relative to the early stage ovarian cancer. In some embodiments, the advanced stage ovarian cancer comprises altered expression of CCNE1 and STAT3 relative to the early stage ovarian cancer. In some embodiments, the advanced stage ovarian cancer comprises altered expression of CCNE1 and VEGF relative to the early stage ovarian cancer.

In some embodiments, the advanced stage ovarian cancer comprises altered expression of CFH and IL-6 relative to the early stage ovarian cancer. In some embodiments, the advanced stage ovarian cancer comprises altered expression of CFH and PZP relative to the early stage ovarian cancer. In some embodiments, the advanced stage ovarian cancer comprises altered expression of CFH and STAT3 relative to the early stage ovarian cancer. In some embodiments, the advanced stage ovarian cancer comprises altered expression of CFH and VEGF relative to the early stage ovarian cancer.

In some embodiments, the advanced stage ovarian cancer comprises altered expression of IL-6 and PZP relative to the early stage ovarian cancer. In some embodiments, the advanced stage ovarian cancer comprises altered expression of IL-6 and STAT3 relative to the early stage ovarian cancer. In some embodiments, the advanced stage ovarian cancer comprises altered expression of IL-6 and VEGF relative to the early stage ovarian cancer.

In some embodiments, the advanced stage ovarian cancer comprises altered expression of PZP and STAT3 relative to the early stage ovarian cancer. In some embodiments, the advanced stage ovarian cancer comprises altered expression of PZP and VEGF relative to the early stage ovarian cancer.

In some embodiments, the advanced stage ovarian cancer comprises altered expression of STAT3 and VEGF relative to the early stage ovarian cancer.

In some embodiments, the advanced stage ovarian cancer comprises altered expression of CCNE1, CFH, and IL-6 relative to the early stage ovarian cancer. In some embodiments, the advanced stage ovarian cancer comprises altered expression of CCNE1, CFH, and PZP relative to the early stage ovarian cancer. In some embodiments, the advanced stage ovarian cancer comprises altered expression of CCNE1, CFH, and STAT3 relative to the early stage ovarian cancer. In some embodiments, the advanced stage ovarian cancer comprises altered expression of CCNE1, CFH, and VEGF relative to the early stage ovarian cancer. In some embodiments, the advanced stage ovarian cancer comprises altered expression of CCNE1, IL-6, and PZP relative to the early stage ovarian cancer. In some embodiments, the advanced stage ovarian cancer comprises altered expression of CCNE1, IL-6, and STAT3 relative to the early stage ovarian cancer. In some embodiments, the advanced stage ovarian cancer comprises altered expression of CCNE1, IL-6, and VEGF relative to the early stage ovarian cancer. In some embodiments, the advanced stage ovarian cancer comprises altered expression of CCNE1, PZP, and STAT3 relative to the early stage ovarian cancer. In some embodiments, the advanced stage ovarian cancer comprises altered expression of CCNE1, PZP, and VEGF relative to the early stage ovarian cancer. In some embodiments, the advanced stage ovarian cancer comprises altered expression of CCNE1, STAT3, and VEGF relative to the early stage ovarian cancer.

In some embodiments, the advanced stage ovarian cancer comprises altered expression of CFH, IL-6, and PZP relative to the early stage ovarian cancer. In some embodiments, the advanced stage ovarian cancer comprises altered expression of CFH, IL-6, and STAT3 relative to the early stage ovarian cancer. In some embodiments, the advanced stage ovarian cancer comprises altered expression of CFH, IL-6, and VEGF relative to the early stage ovarian cancer. In some embodiments, the advanced stage ovarian cancer comprises altered expression of CFH, PZP, and STAT3 relative to the early stage ovarian cancer. In some embodiments, the advanced stage ovarian cancer comprises altered expression of CFH, PZP, and VEGF relative to the early stage ovarian cancer. In some embodiments, the advanced stage ovarian cancer comprises altered expression of CFH, STAT3, and VEGF relative to the early stage ovarian cancer. In some embodiments, the advanced stage ovarian cancer comprises altered expression of IL-6, PZP, and STAT3 relative to the early stage ovarian cancer. In some embodiments, the advanced stage ovarian cancer comprises altered expression of IL-6, PZP, and VEGF relative to the early stage ovarian cancer. In some embodiments, the advanced stage ovarian cancer comprises altered expression of IL-6, STAT3, and VEGF relative to the early stage ovarian cancer. In some embodiments, the advanced stage ovarian cancer comprises altered expression of PZP, STAT3, and VEGF relative to the early stage ovarian cancer.

In some embodiments, the advanced stage ovarian cancer comprises altered expression of CCNE1, CFH, IL-6, and PZP relative to the early stage ovarian cancer. In some embodiments, the advanced stage ovarian cancer comprises altered expression of CCNE1, CFH, IL-6, and STAT3 relative to the early stage ovarian cancer. In some embodiments, the advanced stage ovarian cancer comprises altered expression of CCNE1, CFH, IL-6, and VEGF relative to the early stage ovarian cancer. In some embodiments, the advanced stage ovarian cancer comprises altered expression of CCNE1, CFH, PZP, and STAT3 relative to the early stage ovarian cancer. In some embodiments, the advanced stage ovarian cancer comprises altered expression of CCNE1, CFH, PZP, and VEGF relative to the early stage ovarian cancer. In some embodiments, the advanced stage ovarian cancer comprises altered expression of CCNE1, CFH, STAT3, and VEGF relative to the early stage ovarian cancer. In some embodiments, the advanced stage ovarian cancer comprises altered expression of CCNE1, IL-6, PZP, and STAT3 relative to the early stage ovarian cancer. In some embodiments, the advanced stage ovarian cancer comprises altered expression of CCNE1, IL~6, PZP, and VEGF relative to the early stage ovarian cancer. In some embodiments, the advanced stage ovarian cancer comprises altered expression of CCNE1, IL-6, STAT3, and VEGF relative to the early stage ovarian cancer. In some embodiments, the advanced stage ovarian cancer comprises altered expression of CCNE1, PZP, STAT3, and VEGF relative to the early stage ovarian cancer. In some embodiments, the advanced stage ovarian cancer comprises altered expression of CFH, IL-6, PZP, and STAT3 relative to the early stage ovarian cancer. In some embodiments, the advanced stage ovarian cancer comprises altered expression of CFH, IL-6, PZP, and VEGF relative to the early stage ovarian cancer. In some embodiments, the advanced stage ovarian cancer comprises altered expression of CFH, IL-6, STAT3, and VEGF relative to the early stage ovarian cancer. In some embodiments, the advanced stage ovarian cancer comprises altered expression of CFH, PZP, STAT3, and VEGF relative to the early stage ovarian cancer. In some embodiments, the advanced stage ovarian cancer comprises altered expression of IL-6, PZP, STAT3, and VEGF relative to the early stage ovarian cancer.

In some embodiments, the advanced stage ovarian cancer comprises altered expression of CCNE1, CFH, IL-6, PZP, and STAT3 relative to the early stage ovarian cancer. In some embodiments, the advanced stage ovarian cancer comprises altered expression of CCNE1, CFH, IL-6, PZP, and VEGF relative to the early stage ovarian cancer. In some embodiments, the advanced stage ovarian cancer comprises altered expression of CCNE1, CFH, IL-6, STAT3, and VEGF relative to the early stage ovarian cancer. In some embodiments, the advanced stage ovarian cancer comprises altered expression of CCNE1, CFH, PZP, STAT3, and VEGF relative to the early stage ovarian cancer. In some embodiments, the advanced stage ovarian cancer comprises altered expression of CCNE1, IL-6, PZP, STAT3, and VEGF relative to the early stage ovarian cancer. In some embodiments, the advanced stage ovarian cancer comprises altered expression of CFH, IL-6, PZP, STAT3, and VEGF relative to the early stage ovarian cancer.

In some embodiments, the advanced stage ovarian cancer comprises altered expression of CFH, CCNE1, IL-6, PZP, STAT3, and VEGF relative to the early stage ovarian cancer.

In some embodiments, the method measures at least 1 exosomal protein to about 30 exosomal proteins. In some embodiments, the at least one exosomal proteins are measured in a serum sample.

In some embodiments, the method distinguishes between an early stage HGSOC and an advanced stage HGSOC. In some embodiments, the exosomal proteins are increased in a subject with the early stage HGSOC subject relative to the healthy control subject. In some embodiments, the exosomal proteins are increased in a subject with the advanced stage HGSOC subject relative to the healthy control subject. In some embodiments, the exosomal proteins are differentially expressed in the subject with the early stage HGSOC subject relative to the subject with the advanced stage HGSOC.

In some embodiments, the method further detects a protein not located in an exosome. In some embodiments, the method further detects HE4, MUC16 (CA-125), or combination thereof. In some embodiments, expression of HE4 is detected with the expression of any combination of proteins of any preceding aspect. In some embodiments, the expression of MUC16 (CA-125) is detected with the expression of any combination of proteins of any preceding aspect.

In some embodiments, the at least one exosomal proteins are measured using a protein detection technique selected from liquid chromatography, mass spectrometry, trapped ion mobility spectrometry (TIMS), time-of-flight (TOF) mass spectrometry, enzyme linked immunoassay (ELISA), or combinations thereof.

In some embodiments, the subject diagnosed with the ovarian cancer is administered an anti-cancer therapy. In some embodiments, the method comprises administering an anti-cancer therapy to the subject with an ovarian cancer. In some embodiments, the anti-cancer therapy comprises chemotherapy, immunotherapy, radiation therapy, surgery, palliative care, or combinations thereof.

Anti-cancer agents encompass biotherapeutic anti-cancer agents as well chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SUS416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

In some embodiments, an anti-cancer surgery includes, but is not limited to resect or removal of a tumor.

In some embodiments, the ovarian cancer comprises a high grade serous ovarian carcinoma (HGSOC). In some embodiments, the type of ovarian cancer that is predicted, identified, diagnosed, and/or tracked comprises a serous, endometrioid, mucinous, and clear cell tumors. In some embodiments, the prediction identification, diagnosis, and/or tracking of ovarian cancer includes the predicting, identifying, diagnosing, and/or tracking of a specific stage of the disease such as Stage I (IA, IB or IC), II, III and IV tumors.

In some embodiments, the disclosure relates to creating a report for a physician of the relative levels of the biomarkers and to transmitting such a report by mail, fax, email or otherwise. In some embodiments, a data stream is transmitted via the internet that contains the reports of the biomarker evaluations. In some embodiments, the report includes the prediction, identification, diagnosis, and/or tracking of the presence or absence of ovarian cancer in the subject or the stratified risk of ovarian cancer for the subject, optionally by subtype or stage of cancer.

In some embodiments, the foregoing evaluation of biomarker expression levels is combined for diagnostic purposes with other diagnostic procedures such as gastrointestinal tract evaluation, chest x-ray, HE4 test, CA-125 test, complete blood count, ultrasound or abdominal/pelvic computerized tomography, blood chemistry profile and liver function tests.

In some embodiments, the disclosure relates to the evaluation of samples drawn from a subject who is symptomatic for ovarian cancer or is at high risk for ovarian cancer. In some embodiments, the disclosure relates to subjects who are asymptomatic of ovarian cancer. Symptomatic subjects have one or more of the following: pelvic mass; ascites; abdominal distention; general abdominal discomfort and/or pain (gas, indigestion, pressure, swelling, bloating, cramps); nausea, diarrhea, constipation, or frequent urination; loss of appetite; feeling of fullness even after a light meal; weight gain or loss with no known reason; and abnormal bleeding from the vagina. The levels of biomarkers may be combined with the findings of such symptoms for a diagnosis of ovarian cancer.

Embodiments of the disclosure are highly accurate for determining the presence of ovarian cancer. By “highly accurate” means a sensitivity and a specificity each at least about 85 percent or higher, more preferably at least about 90 percent or 92 percent and most preferably at least about 95 percent or 97 percent accurate. Embodiments of the disclosure further include methods having a sensitivity of at least about 85 percent, 90 percent or 95 percent and a specificity of at least about 55 percent, 65 percent, 75 percent, 85 percent or 90 percent or higher. Some embodiments include methods having a specificity of at least about 85 percent, 90 percent or 95 percent, and a sensitivity of at least about 55 percent, 65 percent, 75 percent, 85 percent or 90 percent or higher.

Embodiments of the disclosure relating sensitivity and specificity are determined for a population of subjects who are symptomatic for ovarian cancer and have ovarian cancer as compared with a control group of subjects who are symptomatic for ovarian cancer but who do not have ovarian cancer. In another embodiment, sensitivity and specificity are determined for a population of subjects who are at increased risk for ovarian cancer and have ovarian cancer as compared with a control group of subjects who are at increased risk for ovarian cancer but who do not have ovarian cancer. In some embodiments, sensitivity and specificity are determined for a population of subjects who are symptomatic for ovarian cancer and have ovarian cancer as compared with a control group of subjects who are not symptomatic for ovarian cancer but who do not have ovarian cancer.

In some aspects, the levels of the biomarkers are evaluated by applying a statistical method such as knowledge discovery engine (KDE™), regression analysis, discriminant analysis, classification tree analysis, random forests, ProteomeQuest®, support vector machine, One R, kNN and heuristic naive Bayes analysis, neural nets and variants thereof.

In some embodiment, a predictive or diagnostic model based on the expression levels of the biomarkers is provided. The model may be in the form of software code, computer readable format or in the form of written instructions for evaluating the relative expression of the biomarkers.

A patient's physician can utilize a report of the biomarker evaluation, in a broader diagnostic context, in order to develop a relatively more complete assessment of the risk that a given patient has ovarian cancer. In making this assessment, a physician will consider the clinical presentation of a patient, which includes symptoms such as a suspicious pelvic mass and/or ascites, abdominal distention and other symptoms without another obvious source of malignancy. The general lab workup for symptomatic patients currently includes a GI evaluation if clinically indicated, chest x-ray, HE4 test, CA-125 test, CBC, ultrasound or abdominal/pelvic CT if clinically indicated, chemistry profile with LFTs and may include a family history evaluation along with genetic marker tests such as BRCA-1 and BRCA-2. (See, generally, the NCCN Clinical Practice Guidelines in Oncology™ for Ovarian Cancer, V.1.2007.)

The present disclosure provides a novel and important additional source of information to assist a physician in stratifying a patient's risk of having ovarian cancer and in planning the next diagnostic steps to take. The present disclosure is also similarly useful in assessing the risk of ovarian cancer in non-symptomatic, high-risk subjects as well as for the general population as a screening tool. It is contemplated that the methods of the present disclosure may be used by clinicians as part of an overall assessment of other predictive and diagnostic indicators.

The present disclosure also provides methods to assess the therapeutic efficacy of existing and candidate chemotherapeutic agents and other types of cancer treatments. As will be appreciated by persons skilled in the art, the relative expression levels of the biomarker panels—or biomarker profiles—are determined as described above, in specimens taken from a subject prior to and again after treatment or, optionally, at progressive stages during treatment. A change in the relative expression of these biomarkers to a non-cancer profile of expression levels (or to a more nearly non-cancer expression profile) or to a stable, non-changing profile of relative biomarker expression levels is interpreted as therapeutic efficacy. Persons skilled in the art will readily understand that a profile of such expressions levels may become diagnostic for cancer or a pre-cancer, pre-malignant condition or simply move toward such a diagnostic profile as the relative ratios of the biomarkers become more like a cancer-related profile than previously.

In another embodiment, the disclosure provides a method for determining whether a subject is developing cancer. The relative levels of expression of the biomarkers are determined in specimens taken from a subject over time, whereby a change in the biomarker expression profile toward a cancer profile is interpreted as a progression toward developing cancer.

The expression levels of the biomarkers of a specimen may be stored electronically once a subject's analysis is completed and recalled for such comparison purposes at a future time.

The present disclosure further provides methods, software products, computer systems and networks, and associated instruments that provide a highly accurate test for ovarian cancer.

The combinations of markers described in this specification provide sensitive, specific and accurate methods for predicting the presence of or detecting ovarian cancer at various stages of its progression. The evaluation of samples as described may also correlate with the presence of a pre-malignant or a pre-clinical condition in a patient. Thus, it is contemplated that the disclosed methods are useful for predicting, identifying, diagnosing, staging, tracking, or detecting the presence of ovarian cancer in a sample, the absence of ovarian cancer in a sample drawn from a subject, the stage of an ovarian cancer, the grade of an ovarian cancer, the benign or malignant nature of an ovarian cancer, the metastatic potential of an ovarian cancer, the histological type of neoplasm associated with the ovarian cancer, the indolence or aggressiveness of the cancer, and other characteristics of ovarian cancer that are relevant to prevention, diagnosis, characterization, and therapy of ovarian cancer in a patient.

It is further contemplated that the methods disclosed are also useful for assessing the efficacy of one or more test agents for inhibiting ovarian cancer, assessing the efficacy of a therapy for ovarian cancer, monitoring the progression of ovarian cancer, selecting an agent or therapy for inhibiting ovarian cancer, monitoring the treatment of a patient afflicted with ovarian cancer, monitoring the inhibition of ovarian cancer in a patient, and assessing the carcinogenic potential of a test compound by evaluating biomarkers of test animals following exposure.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Exosomal Proteins as Novel Biomarkers for Early Detection in High-Grade Serous Ovarian Cancer

Importance: High-grade serous ovarian cancer (HGSOC) accounts for over 75% of all epithelial OC, and has a high mortality rate due to a lack of early detection methods. Many biomarkers have been proposed, however have not been proven to improve detection at an early stage or improved survival in a routine-risk population.

Objective: Identify unique exosomal protein signatures of early-stage HGSOC that serve as early detection biomarkers.

Design: This is a multi-institutional retrospective cohort study. This study was conducted over five years of patient serums samples from women with all stages of HGSOC and healthy controls. Exosomes were isolated from serum samples from patients with and without HGSOC using our novel microfluidics device, an Exo-quick kit and ultracentrifugation. Mass spectrophotometry (1D-LC-MS/MS at 2 different institutions), Proximity Extension assay (PEA) and Ingenuity Pathway Analysis (IPA) were used to identify the exosomal protein profiles that are differentially expressed between patients with and without HGSOC, and between patients with early-versus advanced-stage disease. The sensitivity and specificity of these exosomal proteins as biomarkers for early-stage HGSOC was then evaluated by ELISA and Luminex.

Setting: Specimens were collected from women receiving care (either for their HGSOC or routine preventive care) at multiple institutions across the United States.

Participants: Women with all stages of HGSOC and who had not yet received chemotherapy were included. Controls were defined as those who had no documented adnexal pathology, cancer, or comorbidities.

Main Outcomes and Measures: It was contemplated that exosomal proteins serve as more sensitive and specific biomarkers for the early detection of HGSOC. This example identifies exosomal proteins that were differentially expressed among clinical specimens of healthy controls and women with early- and advanced-stage HGSOC. The sensitivity and specificity of the exosomal proteins identified were also assessed by Receiver Operating Characteristic (ROC) analyses.

Results: A total of 247 serum samples were evaluated. A greater exosome concentration in HGSOC samples compared to controls was observed, and higher concentrations among advanced-compared to early-stage samples were also observed. LC-MS/MS and PEA of serum samples from patients with early-stage HGSOC identified over 20 exosomal proteins that are differentially expressed in HGSOC compared to controls. Among the proteins identified, several were implicated in carcinogenesis pathways by IPA analysis, including STAT3, CCNE1, FAS, SPP24, IL-6, MMP3, NID1, TETN and TTR. On validation with antibody array, several proteins had a high AUC on ROC analysis of early-stage compared to controls or advanced stage including: CFH (0.94), PZP (0.91), CCNE1 (0.83), FAS (0.76), SPP24 (0.76), STAT3 (0.75), and AGRIN (0.74). In comparison, MUC16 (aka CA-125) had an AUC of 0.78 in exosomes but only of 0.42 in whole serum. Furthermore, MUC16 (aka CA-125) was able to distinguish between controls and early- and advanced-stage HGSOC in exosomes but in whole serum was only differentially expressed between controls and advanced-stage disease.

Conclusion and Relevance: Several exosomal proteins are significantly and differentially expressed in the exosomes of patients with early-stage HGSOC compared to controls and patients with advanced disease, and these exosomal proteins serve as early-detection biomarkers for HGSOC. This work has the ability to be readily translated for clinical use and will have a substantial impact by facilitating earlier detection of HGSOC.

### Materials and Methods

Exosome isolation by ultracentrifugation. Exosomes were isolated from 250 μl of cell-free serum. Samples were first thawed on ice and then diluted in 1 ml 1× phosphate-buffered saline (PBS). The diluted serum samples were centrifuged at 400×g for 15 min to remove debris and the supernatants were transferred to a fresh tube and centrifuged at 10,000×g for 30 min to remove large sized microvesicles and apoptotic vesicles. The supernatants were then filtered through 0.22 μm porous membranes followed by a spin at 100,000×g for 90 min at 4 °C to isolate the exosomes. The exosome pellets were then washed in 1 ml of 1×PBS followed by a second step of ultracentrifugation at 100,000×g at 4 °C for 90 min. The final pellet was re-suspended in PBS and the vesicular protein concentration was determined using Direct detect spectrophotometer.

Exosome isolation by commercial kit. The exosomes were isolated from HGSOC or control patient serum samples (100 μl) using Exoquick kit solution according to the manufacturer's (System biosciences) protocol. The resulting exosome pellet was re-suspended in 100 μl of cold PBS or lysis buffer according to the downstream applications it was to be used for.

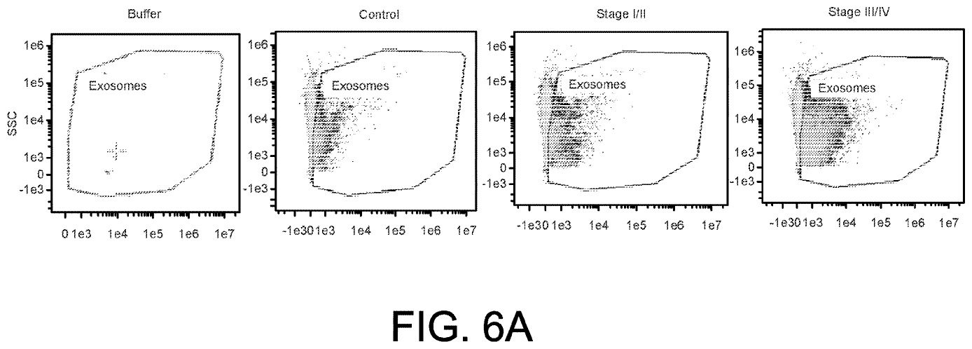

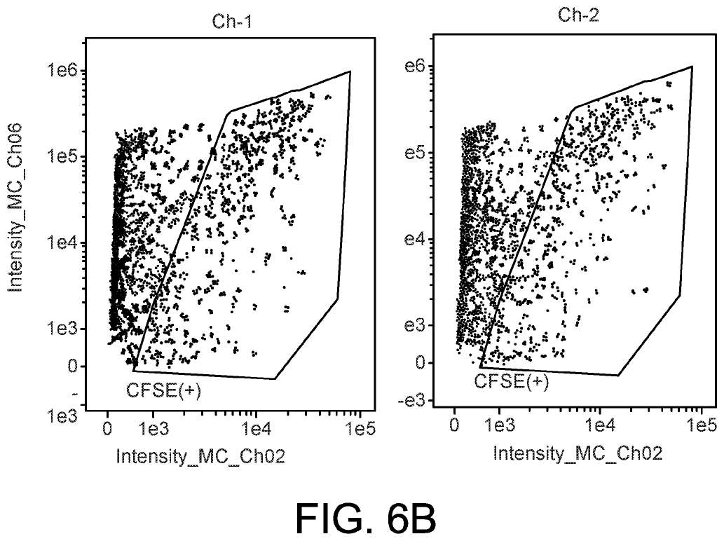

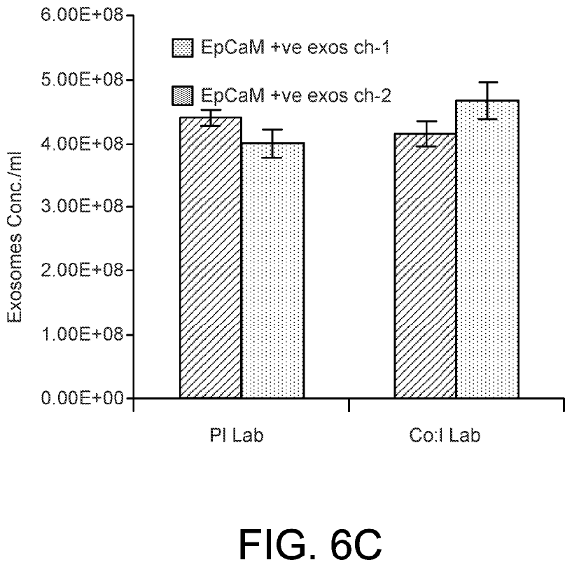

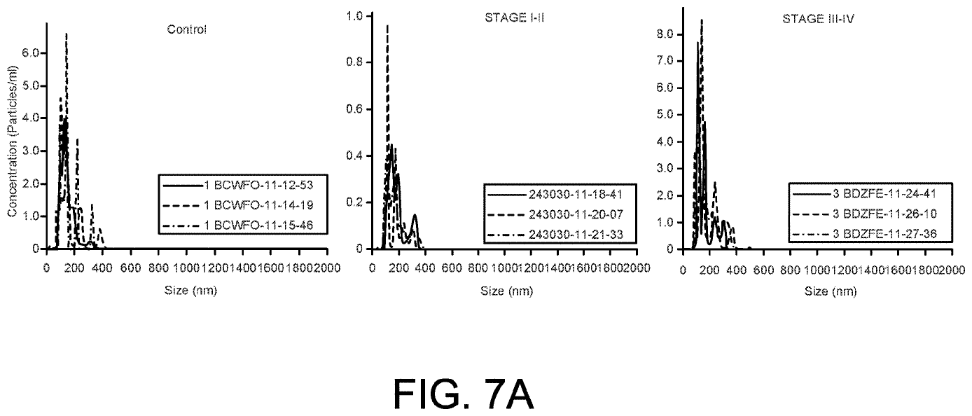

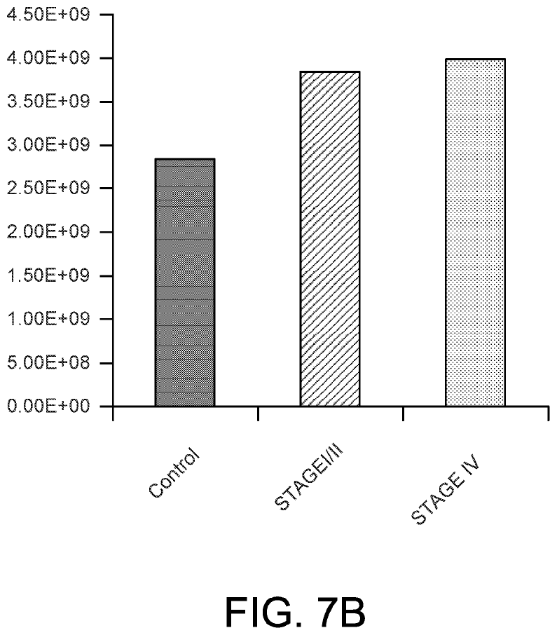

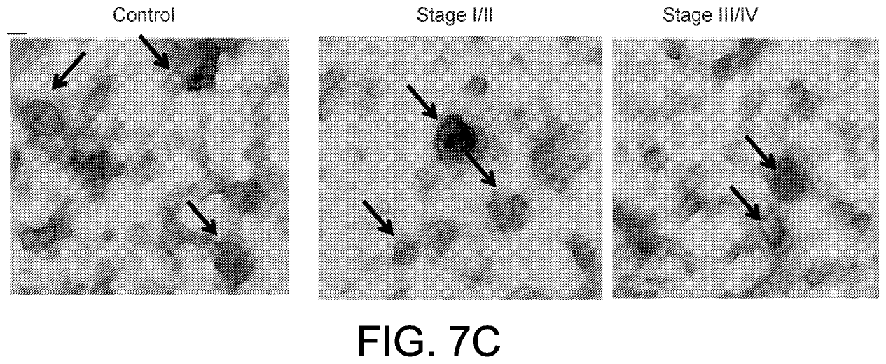

Exosome isolation by microfluidics device (MFD). The process of exosome isolation and confirmation by MFD is shown in FIGS. 6A, 6B, 6C, 7A, 7B, and 7C. The serum samples were thawed on ice and then diluted from 1:5 in 0.22u filtered PBS. The diluted serum flowed through the MED that had been coated with the exosome-specific surface marker antibodies CD9, CD63 and TSG101, and epithelial surface marker EpCAM. Following this, the devices were washed with 0.22u filtered PBS at 25 to 30 μl/min flow rate for 10 min. Exosomes captured on the device were then eluted with 400 μl of Glycine-HCl buffer at 80 μl/min followed by pressurized air through channels to ensure complete collection. The collected exosomes were stored at −80 °C until further downstream processing. Validation of exosome isolation and purification by MFD was performed by testing across 2 different laboratories (FIGS. 6B and 6C) and compared with other standard methods including ultracentrifugation and the commercial Exoquick kit, as shown in FIGS. 6A and 7.

### Exosome Confirmation and Quantification

Nano particle tracking analysis (NTA). After capture and release of the vesicles from the microfluidics device, serum exosomal samples were diluted with PBS (1:4) and (1:10). For reliable NTA measurement, an appropriate exosome sample dilution was used that provides approximately 20-100 particles in the field of view. NTA post-acquisition settings were optimized and kept constant between samples, and each video was then analyzed to give the mean, mode, and median particle size together with an estimated number of particles per mL serum or per 107 cells. Three 10 sec videos were recorded on the NanoSight LM10, which were analyzed using tracking software. The NTA software plots particle size range versus particle number in the sample. 100 nm polystyrene latex microspheres were routinely analyzed to confirm instrument performance.

ImageStream Flow Cytometry (ISF). The ImageStream was equipped with a 60× objective to detect particles in the exosome/extracellular vesicle size range, with a Numerical Aperture (NA) of 0.9 and a resolution of 3 μm²/pixel. Although many of the vesicles are below the size of the pixel resolution, when labelled with fluorescent molecules these smaller vesicles become detectable due to the intensity of the signal, and the width of the core stream is reduced to 7 μm to increase the frequency of in-focus objects. In addition, the speed was set to the lowest setting for maximum resolution. All sheath buffers were filtered with 0.1 μm filters to ensure that there were minimal background particulates. The instrument is equipped with 488 nm (blue), 561 nm (green) and 642 nm (red) lasers to be compatible with the Exo-FITC, PE and AF642 fluorophores and 200 mW was used to increase the number of photons generated per fluorochrome molecule, including the 758 nm laser for scatter measurements.

Scanning electron microscope (SEM). Channels were disassembled and treated with Karnovsky's fixative (2.5% EM-grade glutaraldehyde, 2% formaldehyde and 0.1M sodium phosphate buffer) for 10 min, washed with PBS twice for 5 min, followed by a series of dehydration treatments of 35% ethanol for 10 min, 50% ethanol twice for 10 min, 70% ethanol twice for 10 min, 90% ethanol twice for 10 min, and 100% ethanol four times for 10 min each. Samples were then coated with palladium/gold and imaged with an InLens detector at 3 keV and 100% charge compensation (Zeiss Ultra 55 SEM).

Cro-Transmission electron microscopy (Cryo-TEM). Cryo-TEM specimens of the exosome solutions were prepared using thin-film plunge freezing in a FEI Vitrobot (Mark IV). The vitrified specimens were mounted onto a Gatan 626.DH cryo-holder. Cryo-TEM observation was performed using low-dose mode on a FEI Tecnai F20 TEM.

### Evaluation of Exosome Proteome Profiles in High Grade Serous Ovarian Cancer Compared to Controls.

LC-MS/MS. Protein identification was performed using nano-liquid chromatography-nanospray tandem mass spectrometry (LC/MS/MS) on a Bruker tims-ToF Pro equipped with a CaptiveSpray source operated in positive ion mode. Samples were separated on a C18 reverse phase column (1.6 μm, 250 mm*75 μm IonOpticks) using a Bruker nanoElute UHPLC system. 200 ng peptide was injected for each analysis. Pre-injection the column was equilibrated with 4 column volumes at 800 bar. Mobile phase A was 0.1% Formic Acid in water and acetonitrile (with 0.1% formic acid) was used as mobile phase B. A flow rate of 0.4 μL/min was used. Mobile phase B increased from 2 to 17% over the first 60 minutes, then increased to 25% over the next 10 minutes, further increased to 37% over the next 10 minutes, and finally increased to 80% over 10 minutes and then held at 80% for 10 minutes. MS and MS/MS experiments were recorded over the m/z range 100-1700 and K0 of 0.6-1.6. PASEF was used for all experiments, with the number of PASEF MS/MS scans set to 10. Active exclusion was applied, released after 0.4 minutes, with precursor reconsidered if current intensity/previous intensity was 4.0 or greater.

MyriMatch Database Search and IDPicker Protein Assembly. Raw mass spectrometry data files were searched with the ProteoWizard platform containing the open source software program MyriMatch (v 2.2.8634). The 15 raw data files (three sample sets, each containing 5 biological replicates) were analyzed with the MyriMatch search engine against the complete reviewed Uniprot human database containing 116 common contaminant proteins and the pepXML output files passed to IDPicker (v 3.1.8754.0) for protein inference and assembly. Default database search parameters included precursor and fragment mass tolerances of 10 ppm and 0.5 Da, trypsin/P digestion after Arg and Lys with variable modifications for carbamidomeythlyation of cysteine (CamC, +57.02146) and oxidation of methionine (MetO, +15.99491). Protein inclusion list required two minimum peptides and two peptide spectral matches (PSMs) while the peptide FDR was manually adjusted to produce an overall protein FDR less than 5% (FDR=2.6%).

Proximity Extension Assay (PEA). A pair of oligonucleotide-labeled antibodies (Proseek probes) was allowed to pair-wise bind to the target protein present in the sample in a homogeneous assay, with no need for washing. When the two Proseek probes are in close proximity, a new PCR target sequence is formed by a proximity-dependent DNA polymerization event. The resulting sequence is subsequently detected and quantified using standard real-time PCR. Four internal controls including two incubation controls, one extension control, and one detection control were included in the assay. In addition, three replicates of negative controls were used to calculate the lower limit of detection (LOD) for each protein. Cross-reactive events were not detected with Proseek Multiplex since only matched DNA reporter pairs are amplified with real-time PCR.

### Exosomal Protein Quantification and Analysis

Enzyme-linked immunoassay (ELISA). Exosomes were lysed using RIPA buffer added with protease cocktail inhibitors and incubated at room temperature for 5 min. Protein quantification was performed using the direct detect infrared spectrophotometer or NanoDrop UV-Vis spectrophotometer. The plates were coated with equal concentration of exosome proteins and incubated on a 96-well assay plate for 48 hours, and then probed with the optimized primary and secondary antibody concentration titrated for each protein target. On addition of the TMB substrate solution to the HRP conjugated secondary antibody, blue color develops which corresponds to the target protein concentration. The reaction was then stopped by the addition of 0.2N Sulphuric acid, which turns the reaction mixture to yellow color. Readings were obtained within 10 min at 405 nm.

Trapped Ion Mobility (TIMS)-Time-Of-Flight (TOF)-Mass Spectrometry (TIMS-TOF-MS) Digestion. Exosome pellets were resuspended in 100 μL of 50 mM ammonium bicarbonate solution (ABC) containing 0.1% Rapigest (Waters Corp). They were then vortexed briefly before proceeding to sonication by using the Bioruptor® Pico (Diagenode, Denville, NJ) following the manufacturer's protocol. Sonication temperature was set at 4 C; sonication cycle was set at 30 sec on and 30 sec off. A total of 10 cycles were performed for the cell pellets. Extracts were clarified at 13K rpm in a microcentrifuge and concentration was determined by Qubit. The extracts were digested with trypsin (protein:enzyme 30:1) with a final concentration of 0.1% Rapigest. Briefly, 5 μL of 50 mM ABC containing 5 μg/μL DTT was added and the samples were incubated at 65 °C for 15 min followed by addition of 5 μL of 50 mM ABC containing 15 μg/μL iodoacetamide (incubated at RT for 15 min in the dark) before sequencing grade trypsin was added and digested overnight at 37 °C. The following day, trifluoro acetic acid was added to a final concentration of 0.5% and samples were incubated at 37 °C for 30 min to precipitate the Rapigest. The sample was clarified at 13K rpm for 5 min in a microcentrifuge, dried in a vacufuge and resuspended in 20 μL of 50 mM acetic acid. Peptides were desalted using C18 ziptip and the concentration of the peptides was determined by nanodrop (A280 nm).

Database Searching. RAW Bruker.d mass spectrometry files from all samples (n=12) were converted to mzML with ProteoWizard and OpenMS (v 2.5.0) with an in-house Nextflow script. Converted files were searched on the OpenMS platform with MSGF+ search engine against a reviewed UniProt human proteome (downloaded Sep. 24, 2020) containing the cRAP and MaxQuant contaminant FASTAs. Search parameters included: full trypsin digest, 1 missed cleavage, carbamidomethylation of cysteine as a fixed modification and oxidation of methionine as a variable modification with precursor and fragment mass tolerances of 20 ppm and 0.05 Da. PSM rescoring was performed with Percolator and protein inference was performed with Epifany across all samples, setting peptide and protein false discovery rates to 0.05.

### Results

### Procurement, Processing and Handling of Human Samples

A total of 138 samples were collected from The Ohio State University (OSU), 76 from Inova, 22 from Cedars Sinai, and 14 from Washington University. Of these, there were 75 controls, 74 were early-stage HGSOC and 87 were advanced-stage HGSOC. Samples were collected from patients between the ages of 39 to 81 years of age (Median age range 60.36).

### Exosome Isolation, Confirmation and Quantification

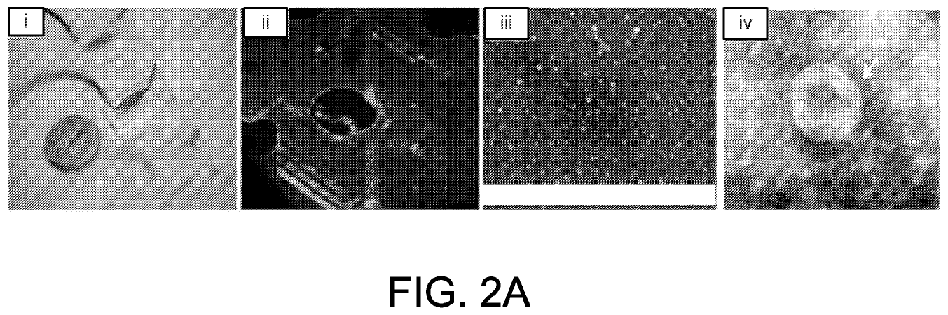

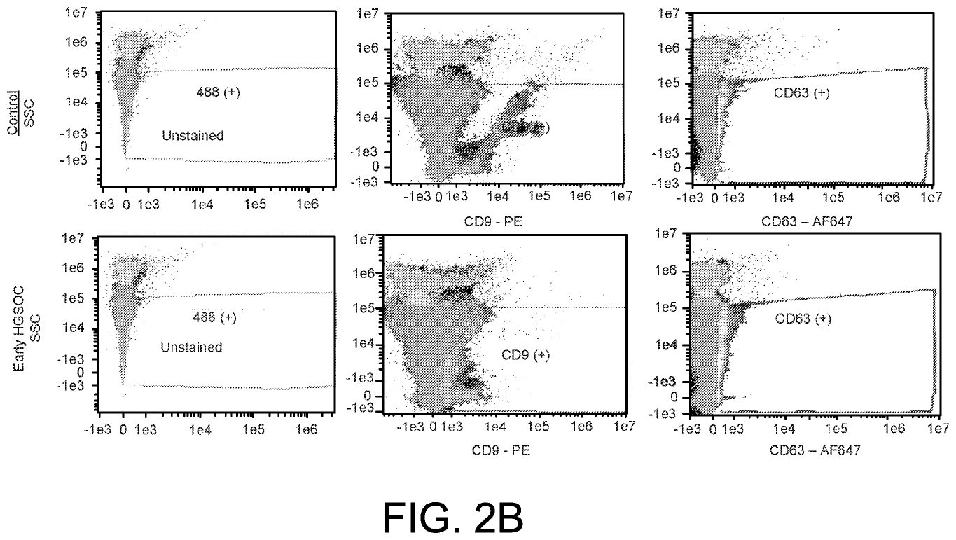

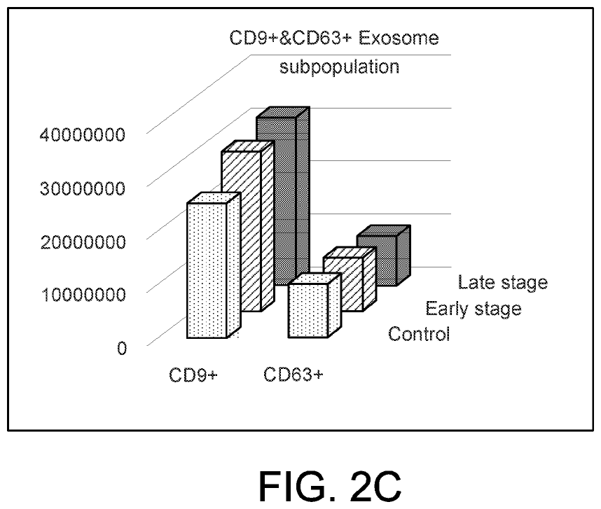

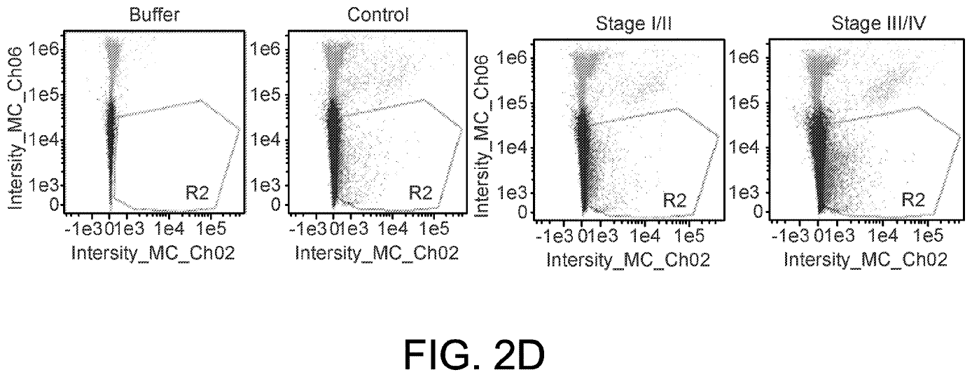

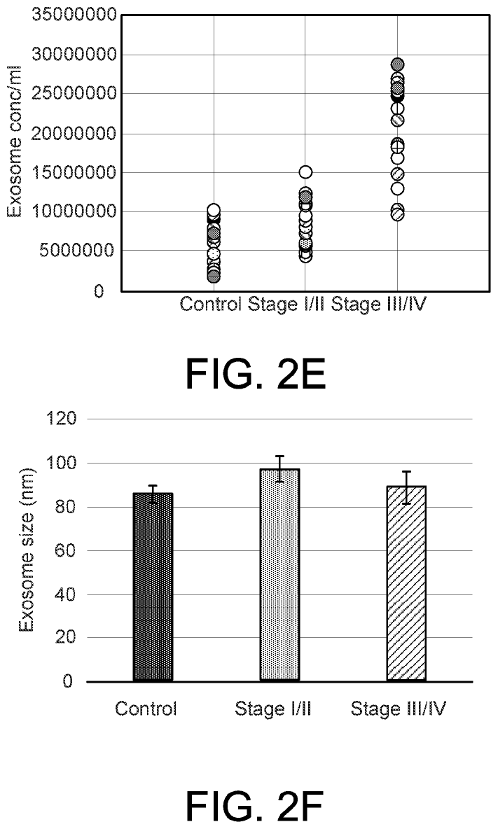

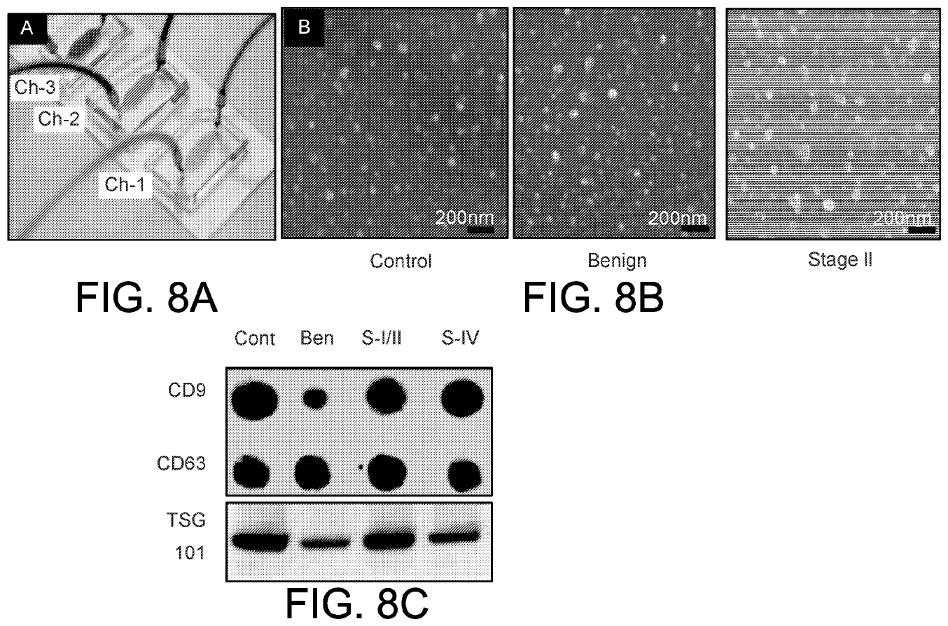

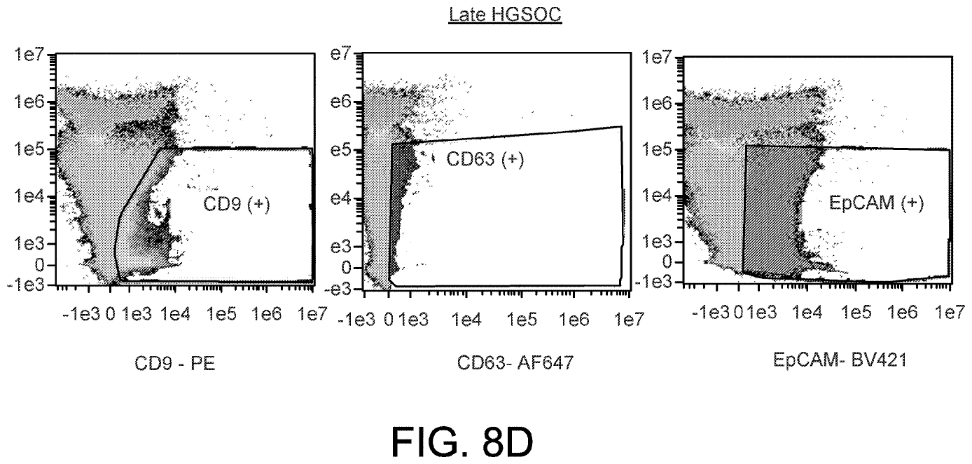

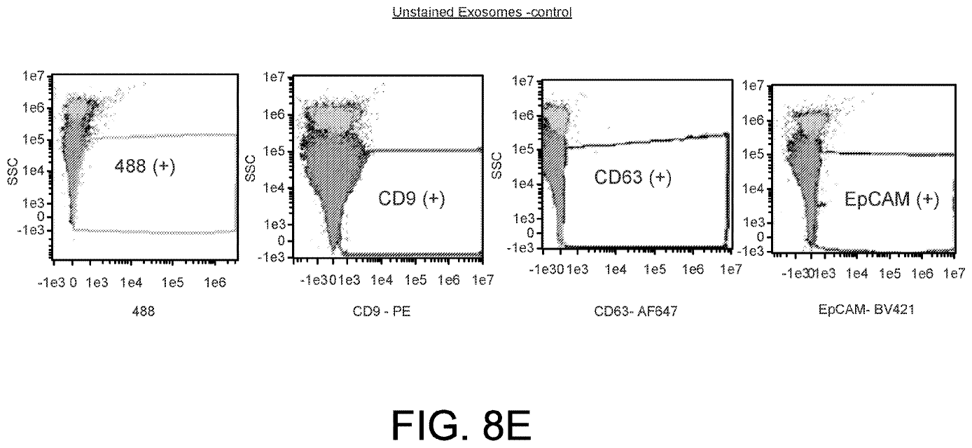

The FITC-Labelled exosomes (green) captured from MFD channels are shown in FIGS. 2Ai, 2Aii, Aiii, and FIG. 8A. Vesicle size and morphology was confirmed by cryo-TEM and SEM (FIGS. 2Aiii, 2Aiv and FIG. 8B). Exosome surface markers CD9, CD63 and TSG101 with epithelial specific markers EpCAM confirmed vesicle lineage from the epithelial cells in controls and HGSOC samples (FIGS. 2B, 2C and FIGS. 8C, 8D, and 8E). When evaluated by ISF and NTA, a sufficient amount of exosome secretion levels and exosomal proteins (500 ng to 5 μg/μl) was detected in controls, early- and advanced-stage HGSOC samples using the novel MFD coated with three different exosome surface markers, including CD9, CD63 and TSG101 (FIGS. 2D, 2E, and 2F). Overall and EpCAM-positive exosomal concentrations were higher in advanced-stage HGSOC samples compared to early-stage HGSOC and control samples (FIGS. 2E, 2F and FIG. 7B). There was no difference in exosome size among groups.

### Evaluation of Exosome Proteome Profiles in High Grade Serous Ovarian Cancer Compared to Controls.

LCMS/MS and Shotgun Proteomics. The subset of proteins elevated in early-stage HGSOC samples relative to control samples based on their difference in expression and fold change are summarized in Table 1. This is termed the discovery dataset of candidate proteins.

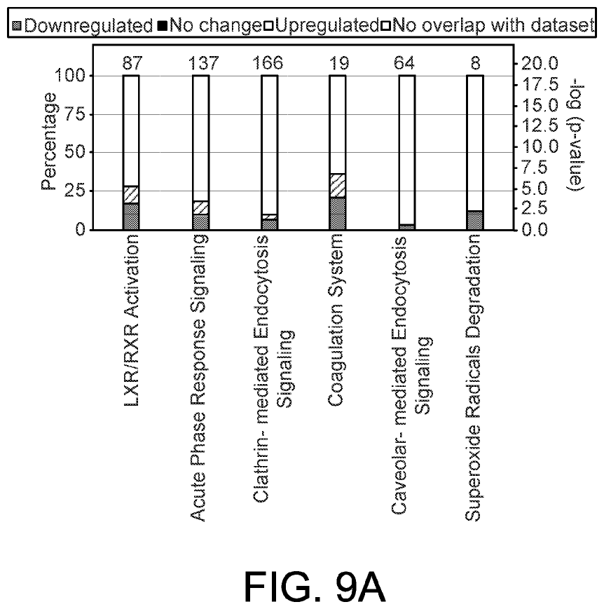

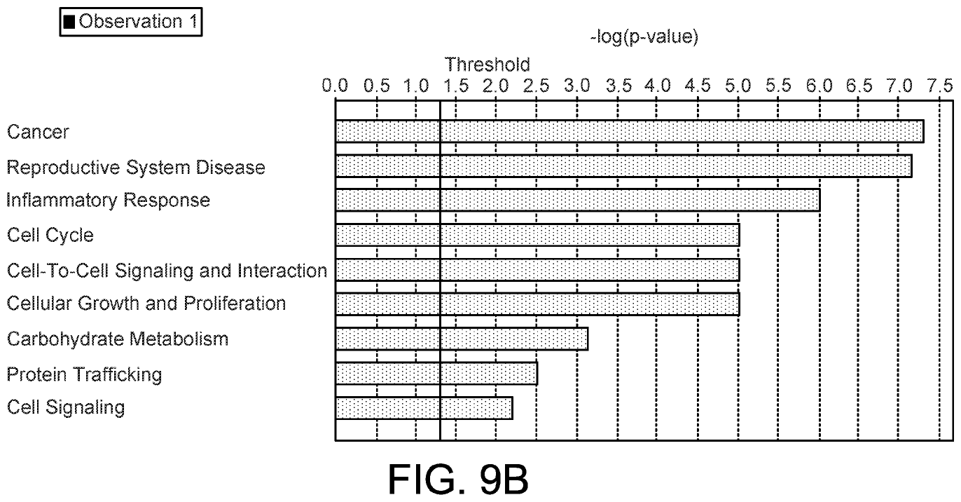

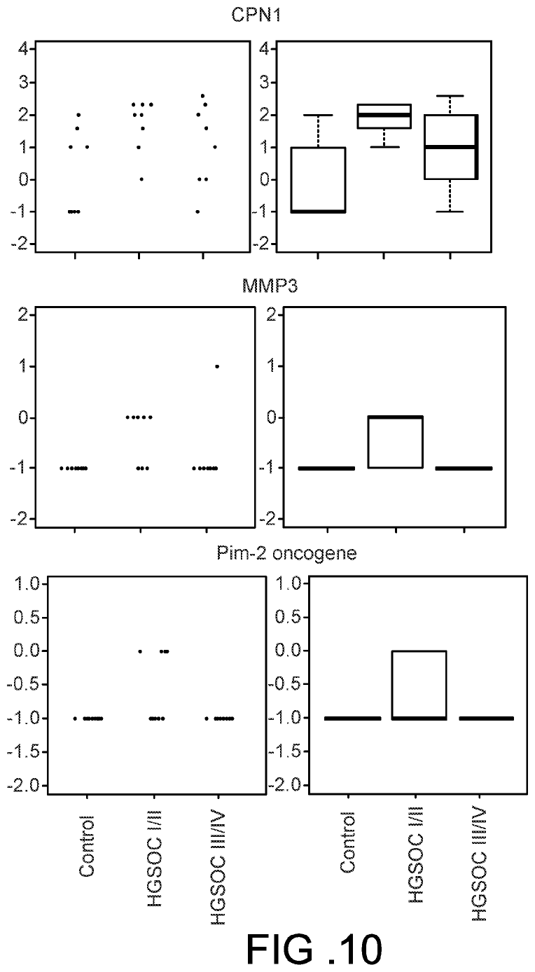

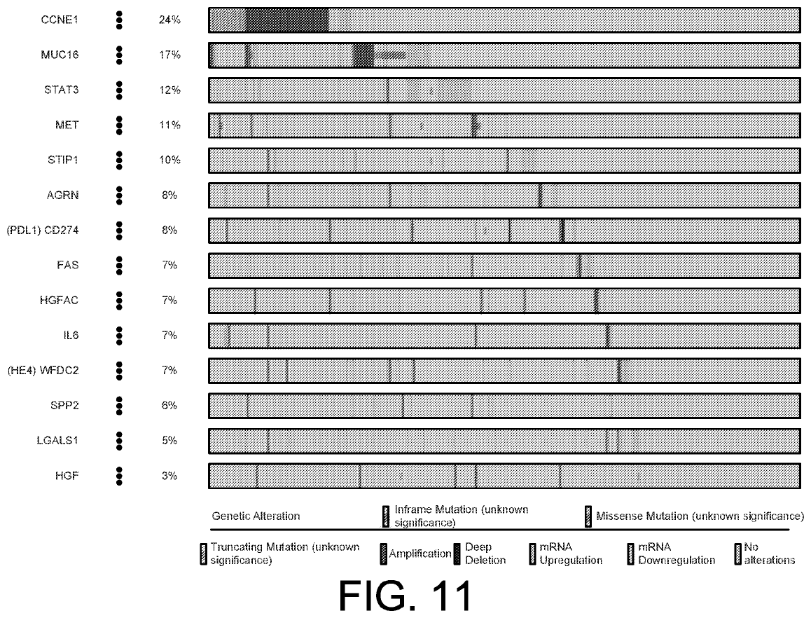

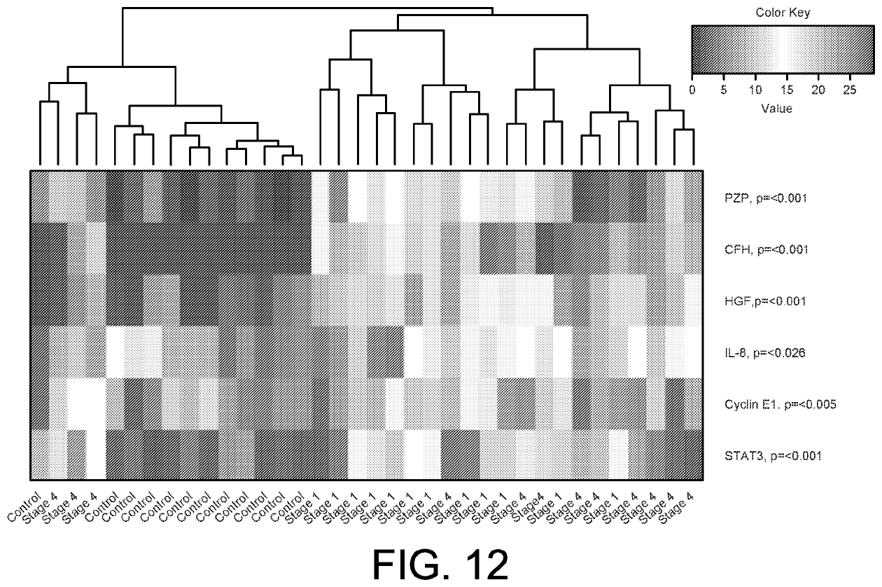

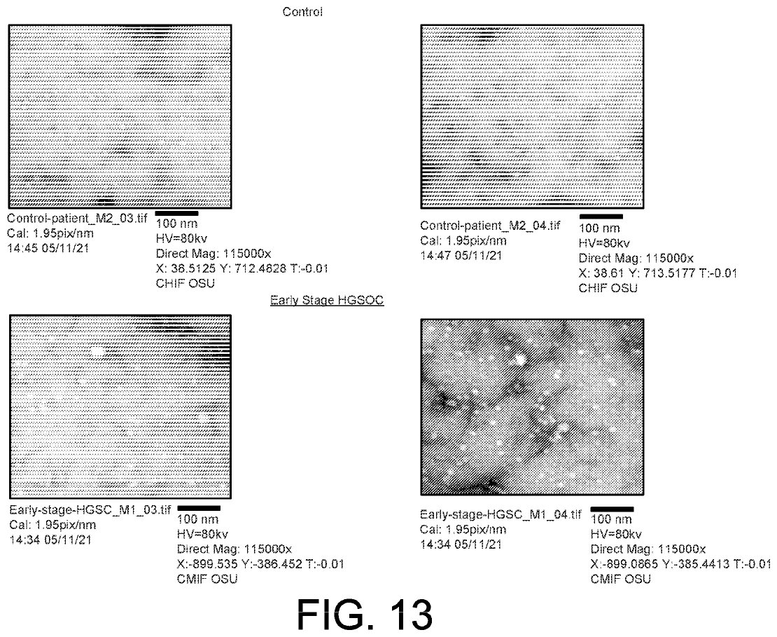

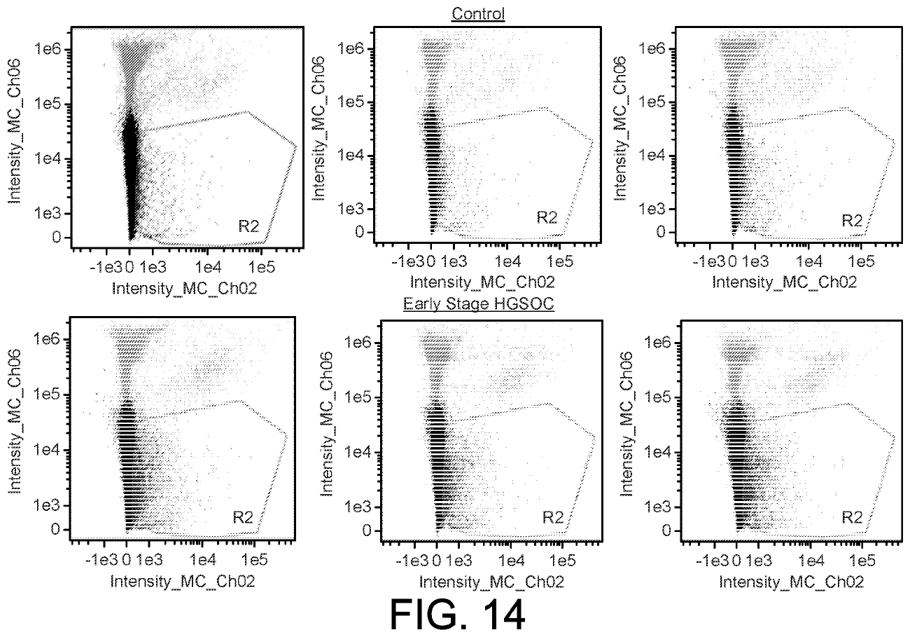

IPA. When the discovery set of candidate proteins were analyzed by IPA, many of those that were differentially expressed were found to have a significant role in cancer regulatory networks, such as cancer cell proliferation, survival, tumor progression and metastasis. These results are shown in FIGS. 9A and 9B. A complete list of candidate proteins identified is in FIG. 10.

PEA. Using PEA, a subset of proteins was again identified that were significantly elevated in early-stage HGSOC serum exosomes compared with controls (Table 5). These include ANG-2, CD40, CXCL1, FAS, GAL1, IL-6, IL-8, MMP-1, PDL1, CFH, Cyclin E, NID1, PZP, and STAT3. It is noted that these proteins can be used alone, or in combination with each other. In one embodiment, one or more of these proteins can be used in combination with HE4.

### Exosomal Protein Quantification and Analysis.

ELISA. Table 2 shows the differential expression of proteins among controls, early, and advanced stage samples in exosomes and whole serum. Exosomal proteins that were significantly elevated in early-stage HGSOC compared with control samples include CFH, CCNE1, FAS, HE4, IL-6, MUC16, pSTAT3, PZP, and STAT3. None of these proteins were found to be differentially expressed in whole serum. Differences in exosomal expression between early-compared to advanced-stage HGSOC were seen in proteins CFH, CCNE1, PZP, STAT3, and VEGF. Again, these differences were not detected in whole serum. Finally, proteins that were differentially expressed in the exosomes of advanced-stage when compared with controls were: CFH, IL-6, MUC16, PZP, pSTAT3, STAT3, and VEGF. In whole serum, MUC16/CA-125 was the only protein that had significantly varied expression among groups, and this was only noted when comparing controls and advanced-stage HGSOC.

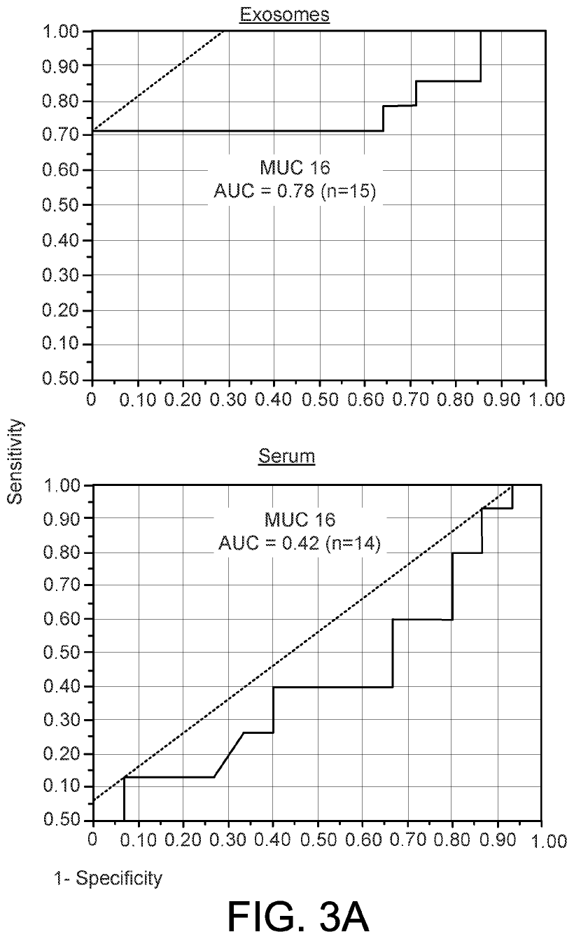

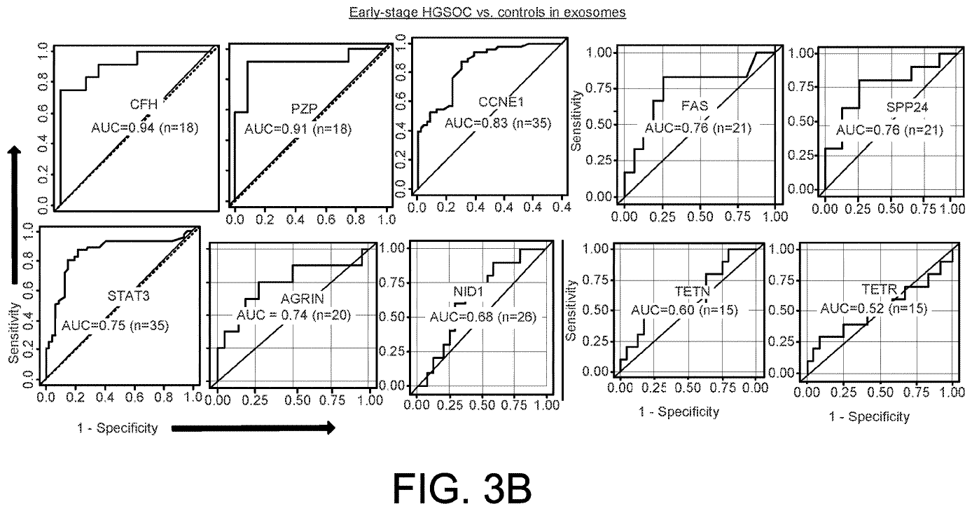

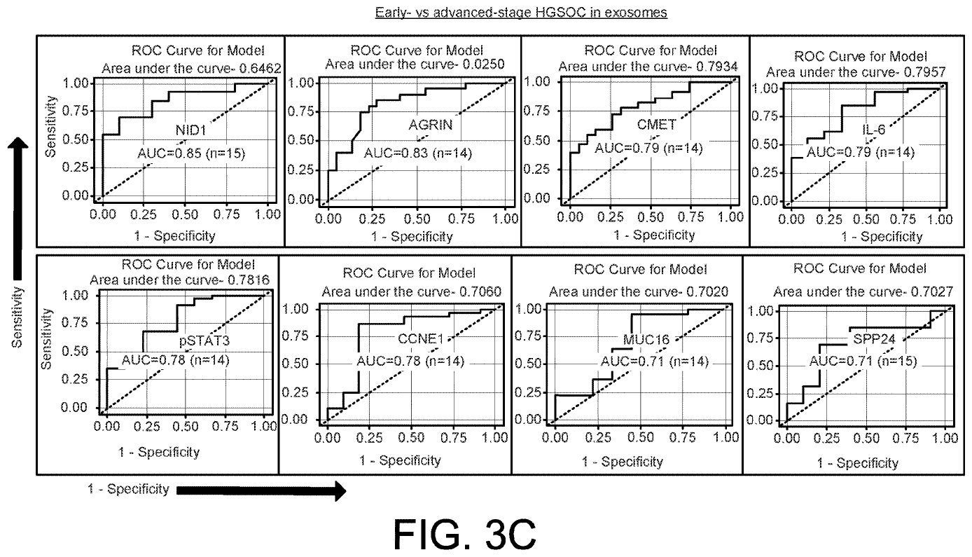

As shown in FIG. 3, sensitivity and specificity analyses by ROC showed differential expression of several individual proteins with AUC's as high as 0.91. In exosomes, MUC16 had an AUC of 0.78, whereas in whole serum the AUC was only 0.42. When comparing early-stage HGSOC and controls, CFH, PCP and CCNE1 had the highest AUC's of 0.94, 0.91 and 0.83, respectively (FIG. 3B). FAS, SPP24, STAT3 and AGRIN also had high AUCs ranging from 0.74 to 0.76 (FIG. 3B). When evaluating early-versus advanced-stage disease, AUC's above 0.70 were seen in AGRIN, CMET, CCNE1, IL-6, MUC16, NID1, pSTAT3, and SPP24 (FIG. 3C).

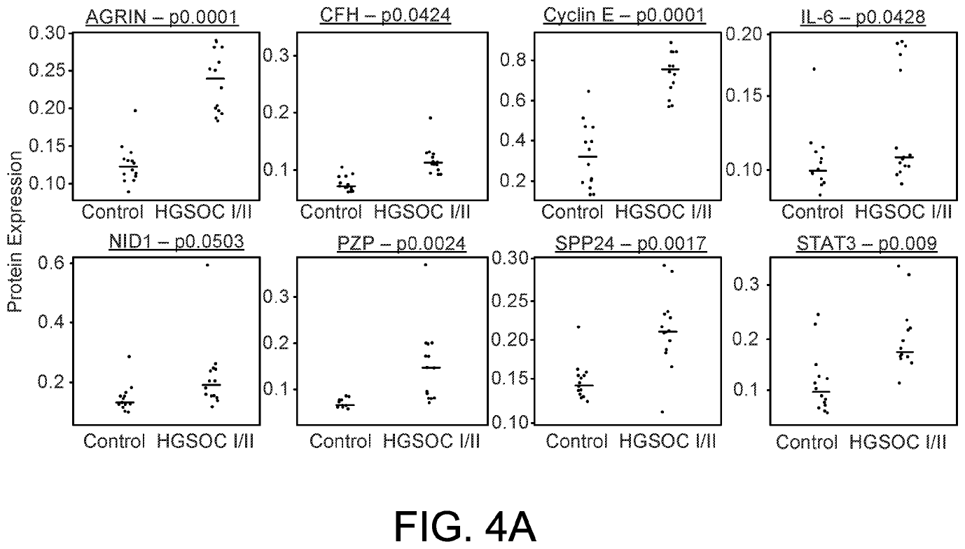

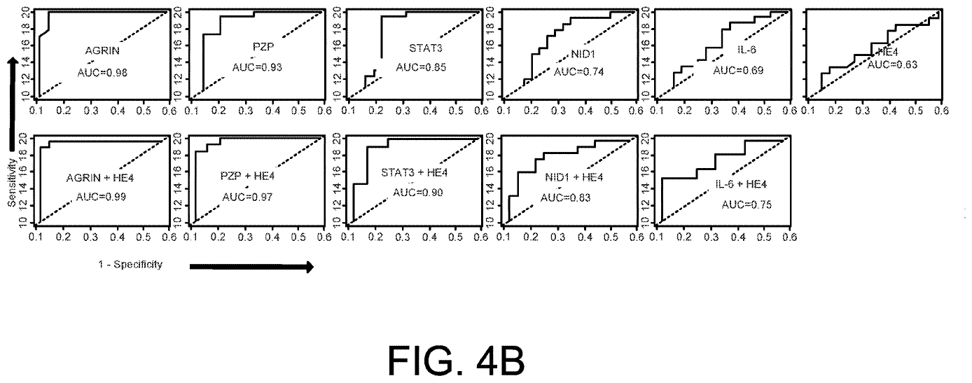

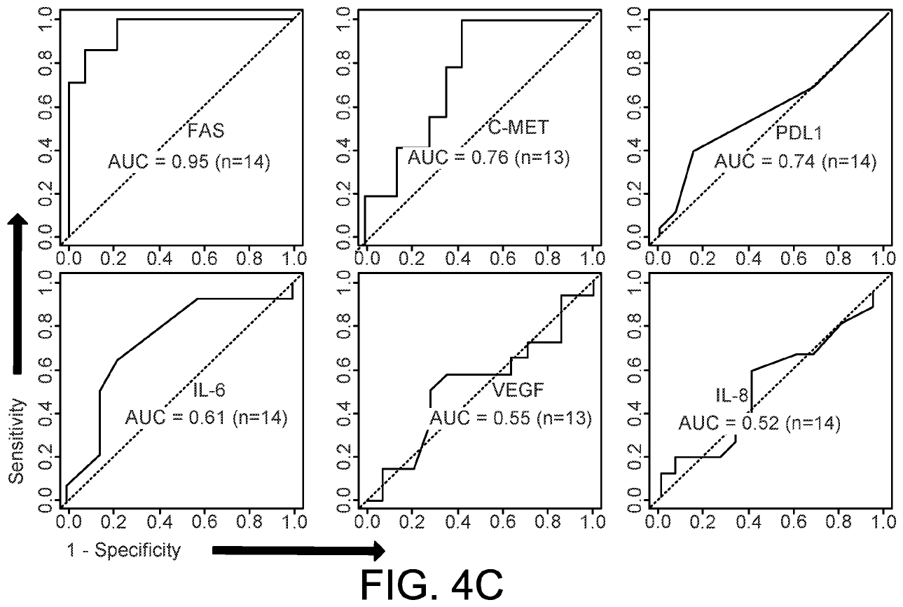

Luminex. Using samples from Washington University, the sensitivity and specificity of the candidate proteins (Table 1) was validated using ELISA and Luminex. ELISA and PEA are quite sensitive and it was contemplated that many of the proteins were detectable in early-stage HGSOC samples. However, if some proteins are not reliably detected or significant in early-stage HGSOC by ELISA, other immune affinity-based methodologies that are more sensitive than ELISA and PEA, such as Luminex, will be used. Differential expression was found between controls and early-stage HGSOC for several proteins, including AGRIN, CFH, CCNE1, IL-6, NID1, PZP, SPP24, and STAT3 (FIG. 4A). ROC analyses resulted in AUCs ranging from 0.52 to 0.95. Specifically FAS, CMET and PDL1 had high AUCs (0.74-0.93) whereas IL-6, VEGF and IL-8 has lower AUCs (0.52-0.61) (FIG. 4C). When assessing these biomarkers in addition to HE4, the AUC was increased only modestly. For example, STAT3 alone had an AUC of 0.85 and this was increased to 0.90 with the addition of HE4.

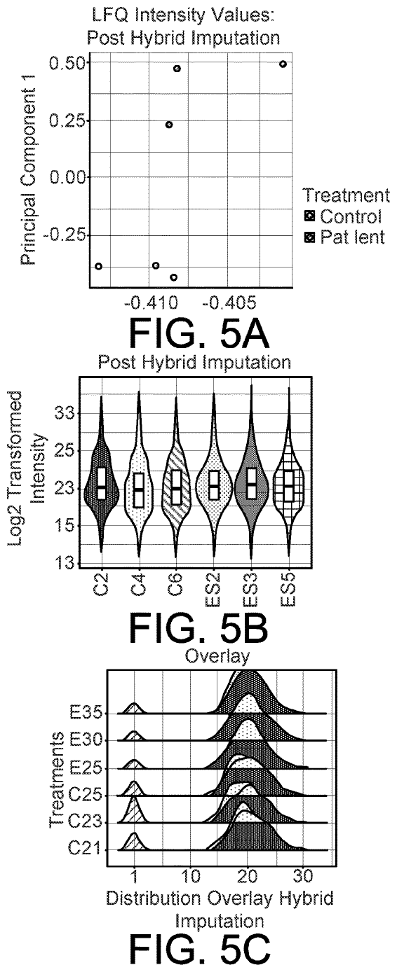

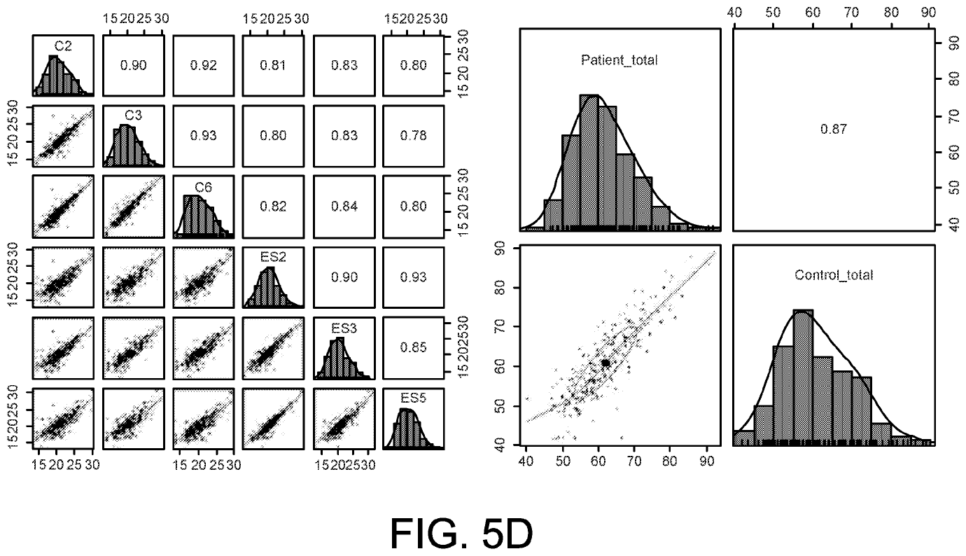

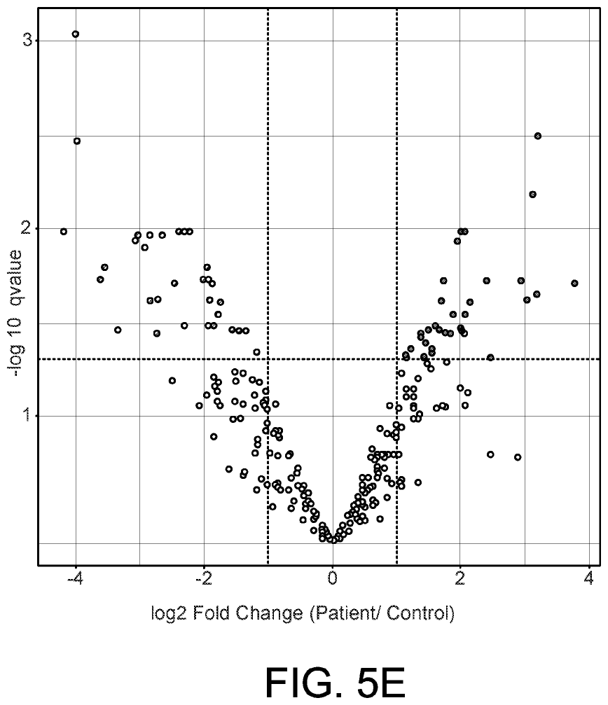

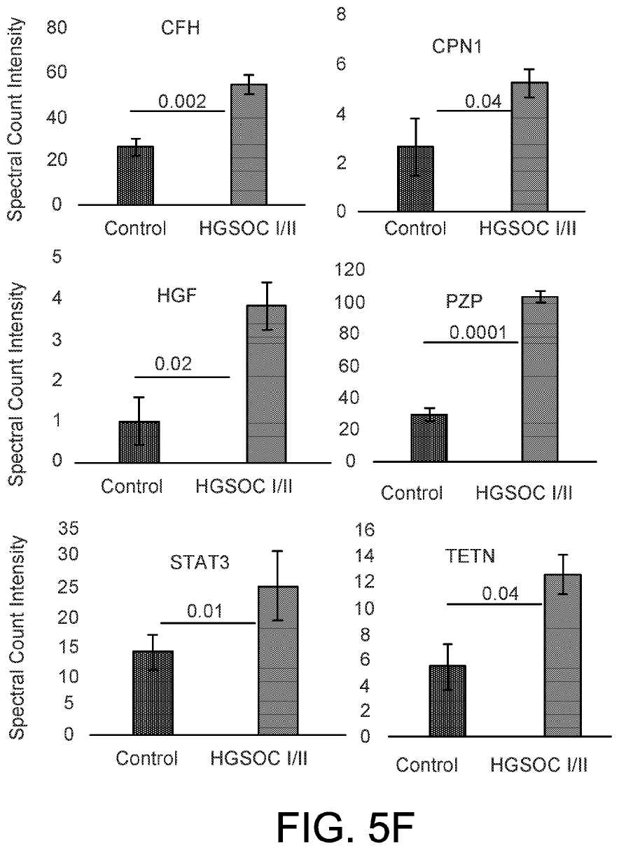

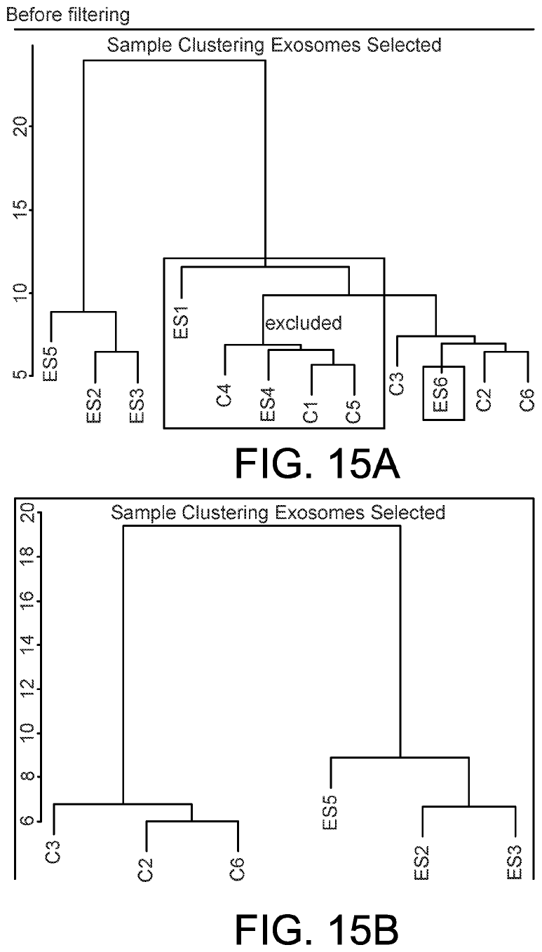

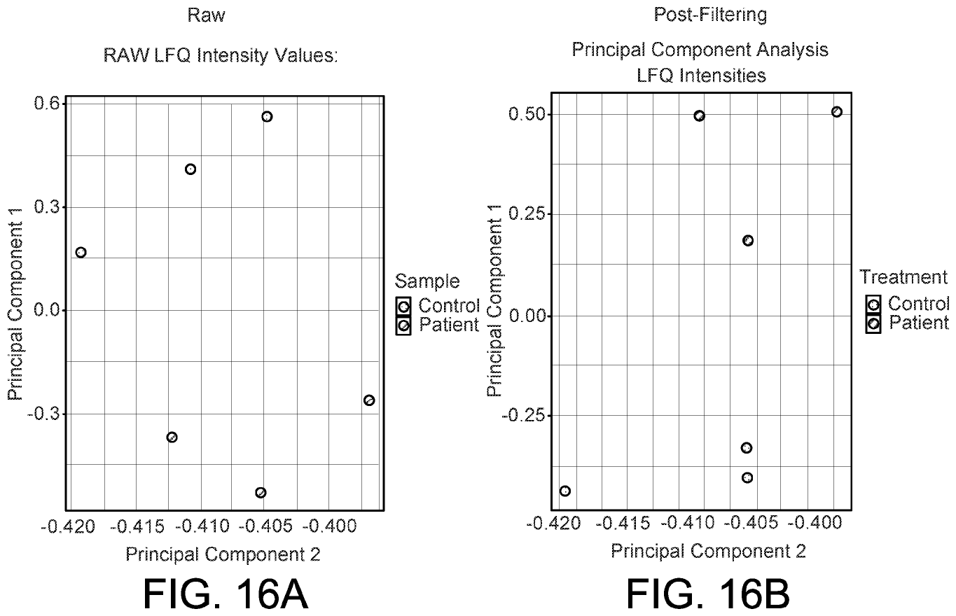

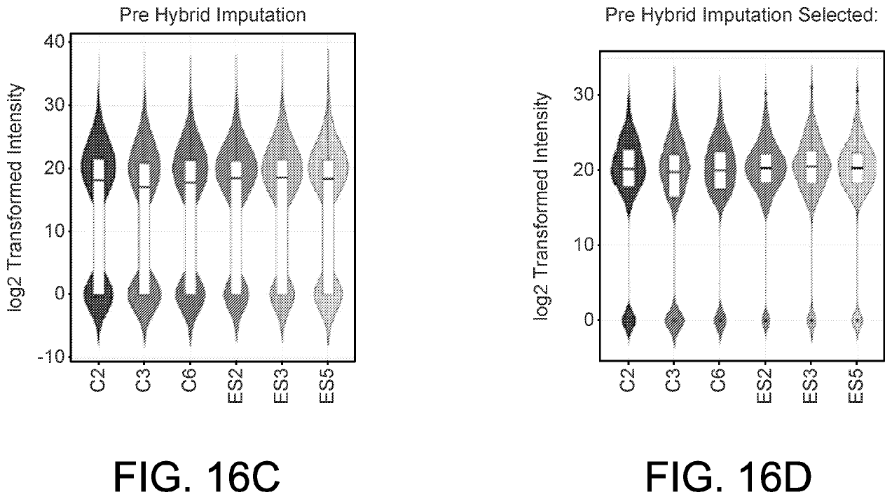

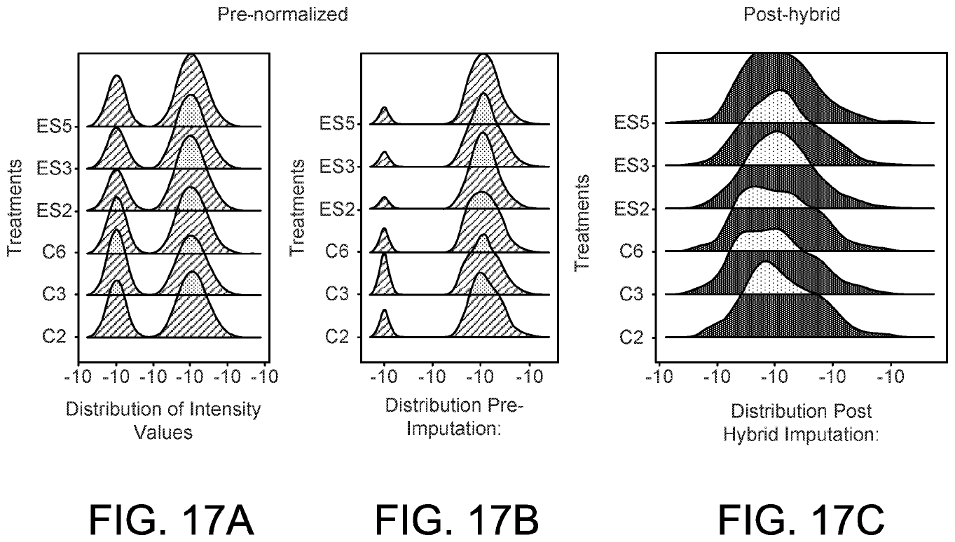

TIMS-TOF-MS. An unbiased clustering analysis on the exosome proteome profiles showed good reproducibility in 50% of the samples (FIGS. 15A and 15B). Further principal component analyses (PCA) demonstrated clustering within the groups while showing the significant variability of the cancer proteome and control exosome indicating a clear separation across the two groups (FIG. 5A) at a 95% Confidence Interval. The spectral count distribution of the proteins with density is shown across the samples by violin plots (FIG. 5B). The tims TOF identified 533 proteins based on the spectral count and 426 proteins based on their relative abundance overall. The differential expression of proteins was identified after filtering lowly expressed proteins and imputing the missing values (FIG. 5C) by multiple imputation methods (FIGS. 16 and 17) with respect to relative abundance. The scatter plot matrices depicts a high degree of correlation and similarity of identified proteins within the control and HGSOC samples and the total abundance across the two groups (FIG. 5D). Quantile normalization of the proteins were done and 73 significant proteins at p-value and significant proteins at q-value determined by exact test (Table 7). With the exclusion of the “outliers” tims TOF showed 92 protein ID's in DE analysis significant at p-value and 73 proteins significant at q-value (Table 7). The Volcano plot (FIG. 5E) represents the differentially expressed proteins in the exosomes (black dashed line) at a significance threshold (q-value <0.05) while the blue dashed lines represents the fold-change < or >2 (log FC>1, log FC<1). The spectral intensity variation with statistical significance for the selected proteins from the DE list in controls versus early-stage HGSOC exosomes are shown in Figure SF.

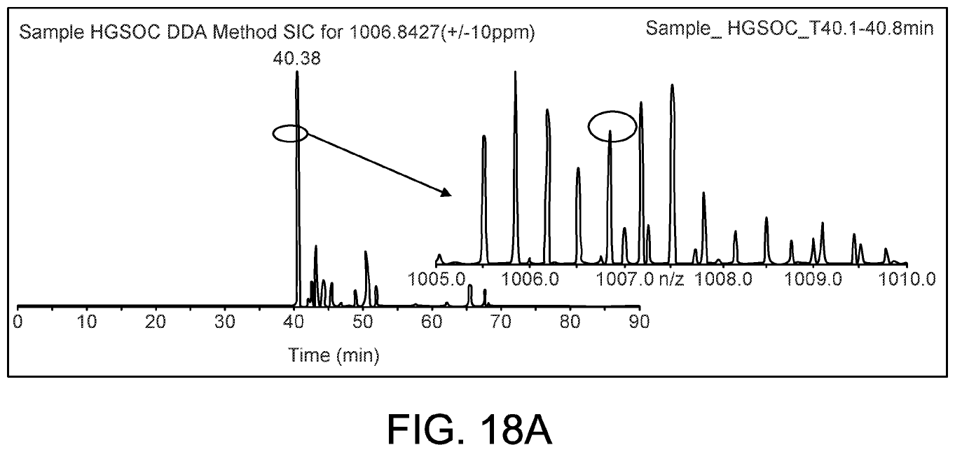

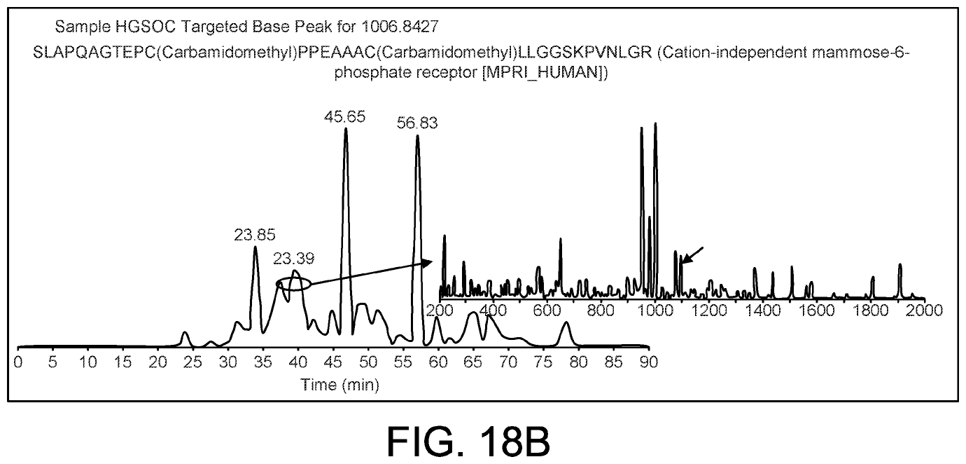

PRM. Identified peptides from the LC-MS/MS experiments (Table 1) were evaluated by a targeted proteomics using Parallel Reaction Monitoring (PRM), as shown in FIG. 17. The PRM data (FIGS. 18A and 18B) showed the signaling average at retention time ~40 min generated inserted MS/MS spectrum, which was identified as SLAPQAGTEPC (SEQ ID NO: 1) (Carbamidomethyl), PPEAAAC (SEQ ID NO: 2) (Carbamidomethyl), and LLGGSKPVNLGR (SEQ ID NO: 3) from human Cation-independent mannose-6-phosphate receptor.

### Discussion

More than 20 exosome-derived serum proteins were identified that are differentially expressed among patients with early-stage HGSOC relative to controls and women with advanced HGSOC (Table 1). In whole serum, only MUC16 (also known as CA-125) could distinguish between any of the groups (controls, early- and advanced-stage HGSOC), and notably could only distinguish between controls and advanced-stage HGSOC. This is consistent with clinical practice, in which CA-125 has low sensitivity (25-61%) and specificity (61-96%) for early-stage disease.

In contrast, several of the exosomal proteins identified herein were able to distinguish between all of the comparator groups (control vs early-stage HGSOC, early-vs advanced-stage HGSOC, and control vs advanced-stage HGSOC), including CFH, PZP, and STAT3. Several other proteins had significantly different expression between some but not all of the comparator groups. When assessing the performance of these same proteins by ROC analyses in two cohorts, several proteins had a high AUC when comparing controls and early-stage HGSOC: AGRIN (0.98), CFH (AUC 0.94), PZP (AUC 0.91 and 0.93), CCNE1 (AUC 0.83), FAS (AUC 0.76 and 0.95), and STAT3 (AUC 0.75 and 0.85). When assessing these proteins in combination with clinically available biomarkers, the addition of HE4 increased the AUC only modestly, likely owing to the fact that each of the exosomal proteins identified already outperformed the currently available biomarker proteins. These findings show there is unique exosomal protein cargo in serum from women with HGSOC that is able to distinguish between stages of disease.

Some of the identified proteins are implicated in ovarian cancer while others have been reportedly involved in cancer pathogenesis in other disease sites but not specifically in ovarian cancer. To the best of one's current knowledge, this is the first report of significant elevation of proteins CFH, NID1, PZP and SPP24 in the serum exosomes of patients with HGSOC. The mechanisms behind these protein levels in patients with ovarian and other cancers have yet to be determined. The biologic functions of these differentially expressed of CCNE1 proteins and their putative roles in pathways associated with ovarian cancer initiation or carcinogenesis have previously been discussed. In addition, CCNE1 gene amplification occurs in approximately 20% of HGSOC and is associated with oncogenic driver in serous ovarian carcinoma, platinum resistance and overall poor prognosis. It has also been shown that CCNE1 expression promotes an accumulation of DNA damage and altered transcription of DNA damage response genes related to DNA replication stress in fallopian tube secretory epithelial cells (FTSECs), showing CCNE1 drives malignant transformation in FTSECs that are the site of origin of serous ovarian carcinomas.

Pregnancy zone protein (PZP) was identified as a novel prognosis biomarker of liver cancer and plays a vital role in the regulation and recruitment of tumor-infiltrating immune cells (TIICs) in the liver cancer immune microenvironment. CFH is expressed in various cancer tissues including ovarian cancer. Recent reports showed that CFH expression is a novel biomarker for lung cancer and that it plays a key role in the resistance of complement-mediated lysis in various cancer cells.

It has been previously shown that overexpression of STAT3 drives FTSECs to transform into HGSOC and that diminishing STAT3 levels using shRNA in established HGSOC reduces tumor growth in vivo. Moreover, activation of STAT3 is observed in more than 70% of human HGSOCs and is associated with tumor progression and decreased survival. These findings provide strength to the argument that FTSECs serves as precursors to HGSOC, and that activation of STAT3 is an important driver of this process. Although the oncogenic function of STAT3 is well established in ovarian cancer, and evidence supports a strong relationship between its expression and early cell transformation, to one's knowledge exosomal STAT3 expression has not previously been proposed as a clinical biomarker.

Collectively, the findings herein show that there is a unique exosomal profile in early-stage HGSOC compared to controls or advanced-stage disease. The exosomal proteins identified were evaluated using different techniques and in samples from various institutions, and were consistently better able to identify early-stage disease compared to biomarkers that are currently clinically available. It should be noted that most women with HGSOC are diagnosed at advanced stages and are not typically enrolled in a tissue banking protocol prior to cancer diagnosis or receiving treatment, limiting availability of early-stage HGSOC specimens. Nonetheless, this example has one of the largest early-stage sample sizes for HGSOC biomarker studies published to date. In addition, the histology samples used herein are early-stage samples from women with HGSOC, whereas prior studies often included low-grade or borderline histologies, which have a different tumor biology and prognosis (JCC 2010). Any issues with sample storage and sample degradation was addressed by limiting the number of freeze-thaw cycles and evaluation of candidate proteins among different subsets of serums samples.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Candidate proteins upregulated in exosomes
from HGSOC early stage compared to
control patient samples by LC-MS/MS.

| Proteins | p < value |
|---|---|
| AGRIN | 0.02 |
| CPNI | 0.005 |
| CFH | 0.01 |
| CMET | 0.003 |
| Cyto P450 2E1 | 0.01 |
| CXCL3 | 0.002 |
| FAS | 0.002 |
| G6-MP | 0.01 |
| HGF | 0.02 |
| IL-6 | 0.009 |
| IL-8 | 0.01 |
| MAPK | 0.001 |
| MMP3 | 0.003 |
| NID1 | 0.001 |
| PDL1 | 0.001 |
| Pim-2 oncogene | 0.03 |
| PZP | 0.02 |
| SPP24 | 0.05 |
| STAT3 | 0.001 |
| TETN | 0.05 |
| TETR | 0.01 |
| VEGFA | 0.03 |

TABLE 2
Candidate protein expression in exosomes and whole serum across controls, early and
advanced stage HGSOC by antibody array

| Did Not | EXOSOMES (p-value) | | | WHOLE SERUM (p-value) | | |
|---|---|---|---|---|---|---|
| Receive | Control v. | Early v. | Control v. | Control v. | Early v. | Control v. |
| Chemo | Early | Advanced | Advanced | Early | Advanced | Advanced |
| MUC16 | 0.004 | 0.559 | 0.002 | 0.153 | 0.700 | 0.0001 |
| HE4 | 0.057 | — | — | 0.014 | — | — |
| AGRIN | 0.225 | 0.261 | 0.076 | 0.970 | 0.806 | 0.918 |
| CFH | 0.003 | 0.01 | 0.005 | 0.4558 | — | — |
| CCNE1 | 0.004 | 0.033 | 0.396 | 0.622 | 0.856 | 0.713 |
| CMET | 0.331 | 0.941 | 0.404 | 0.356 | 0.870 | 0.489 |
| FAS | 0.008 | 0.696 | 0.076 | 0.353 | 0.968 | 0.395 |
| IL6 | 0.004 | 0.934 | 0.031 | 0.591 | 0.975 | 0.650 |
| PDL1 | 0.651 | 0.698 | 0.819 | 0.572 | 0.970 | 0.619 |
| PZP | 0.006 | 0.02 | 0.015 | 0.832 | — | — |
| pSTAT3 | 0.01 | 0.449 | 0.02 | 0.370 | 0.8367 | 0.494 |
| STAT3 | 0.02 | 0.015 | 0.011 | 0.278 | 0.988 | 0.320 |
| STIP1 | 0.316 | 0.768 | 0.286 | 0.845 | 0.885 | 0.924 |
| VEGF | 0.26 | 0.02 | 0.007 | — | — | — |

TABLE 3
Existing patient serum samples (early, late stage HGSOC and control
(without cancer) used for this study from PI and collaborators institutions.

| Sample | | | STAGE | STAGE |
|---|---|---|---|---|
| source | Characteristic | Control | I/II | III/IV |
| The | Number | 65 | 30 | 43 |
| Ohio | Mean of Age in years | 56 | 61 | 60 |
| State | range | (26-79) | (53-78) | (45-78) |
| University | Chemo prior to blood | NA | 18 | 23 |
| | draw (n) | | (12 unknown) | (20 unknown) |
| | Histology | NA | HGSOC | HGSOC |
| INOVA | Number | 15 | 21 | 40 |
| | Mean of Age in years | 54 | 58 | 63 |
| | range | (30-76) | (41-84) | (40-83) |
| | Chemo prior to blood | NA | 21 | 26 |
| | draw (n) | | | (14 unknown) |
| | Histology | NA | HGSOC | HGSOC |
| Washington | Number | NA | 14 | NA |
| University | Mean of Age in years | NA | 59 | NA |
| | range | | (40-72) | |
| | Chemo prior to blood | NA | 11 | NA |
| | draw (n) | | (3 unknown) | |
| | Histology | NA | HGSOC | NA |
| Cedar's | Number | 5 | 12 | 5 |
| Sinai | Mean of Age in years | 59 | 62 | 64 |
| Cancer | range | (28-76) | (39-81) | (46-81) |
| Institute | Chemo prior to blood | NA | unknown | unknown |
| | draw (n) | | | |
| | Histology | NA | HGSOC | HGSOC |

TABLE 4
Serum exosomal proteins that were
differentially expressed in early stage HGSOC
samples compared to benign samples by PEA (n = 12).

| Protein | p-value |
|---|---|
| ANG-2 | 0.001 |
| CD40 | 0.016 |
| CXCL1 | 0.041 |
| EGF | 0.046 |
| FAS | 0.037 |
| GAL1 | 0.015 |
| IL-6 | 0.006 |
| IL-8 | 0.026 |
| MMP-1 | 0.013 |
| PDL1 | 0.001 |
| STAT3 | 0.001 |

TABLE 5
Candidate proteins sensitivity and specificity by Area Under Curve (AUC).

| | Individual Protein | Combination with HE4 |
|---|---|---|
| Protein | (AUC) | (AUC) |
| HE4 | 0.63 | |
| AGRIN | 0.98 | 0.99 |
| CFH | 0.96 | 0.98 |
| CYCLIN E | 0.93 | 0.99 |
| IL6 | 0.69 | 0.75 |
| NID1 | 0.74 | 0.83 |
| PZP | 0.93 | 0.97 |
| SPP24 | 0.89 | 0.90 |
| pSTAT3 | 0.96 | 0.97 |
| STAT3 | 0.85 | 0.90 |

TABLE 6
Comparison of the number of proteins identified based on spectral
count (SC) and abundance (Abun) after filtering lowly expressed
proteins, showing significant proteins at p-value and at q-value. The
tims TOF identified more proteins overall based on the Abundance and
the exclusion of the “outliers” helped the statistics in DE analysis at q-value.

| | | | Sig. | Sig. |
|---|---|---|---|---|
| Analysis | Protein | Proteins after | protein ID's | protein ID's |
| method | ID's | filtering | at pValue | at q Value |
| tims_SC_All | 533 | 250 | 24 | 1 |
| tims_SC_Sel | 533 | 245 | 38 | 0 |
| tims_Abun_All | 426 | 288 | 74 | 16 |
| tims_Abun_Sel | 426 | 292 | 92 | 73 |

TABLE 7
Candidate proteins compression with
prior chemo vs chemotherapy treated patients.

| Protein | Prior Chemo | Chemo |
|---|---|---|
| FAS | 0.008 | 0.004 |
| IL-6 | 0.004 | 0.021 |
| C-MET | 0.331 | 0.441 |
| PDL1 | 0.651 | 0.494 |
| VEGF | 0.26 | 0.064 |

1. A method of diagnosing a subject with a high grade serous ovarian carcinoma (HGSOC), the method comprising measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, phosphorylated STAT3 (pSTAT3), TETN, TETR, or VEGF1, wherein the subject comprises an increased amount of the one or more exosomal proteins relative to a healthy control subject, and wherein the increased amount of the one or more exosomal proteins identifies an ovarian cancer.

2. The method of claim 1, wherein the method measures at least 1 exosomal protein to about 30 exosomal proteins.

3. The method of claim 1, wherein the at least one exosomal proteins are measured in a serum sample.

4. The method of claim 1, wherein the method distinguishes between an early stage HGSOC and an advanced stage HGSOC.

5. The method of claim 1, wherein the exosomal proteins are increased in a subject with the early stage HGSOC subject relative to the healthy control subject or relative to a subject with advanced stage HGSOC.

6. The method of claim 1, wherein the exosomal proteins are increased in a subject with the advanced stage HGSOC subject relative to the healthy control subject.

7. (canceled)

8. The method of claim 1, wherein the method further detects a protein not located in an exosome.

9. The method of claim 1, wherein the method further detects HE4, MUC16 (CA-125), or combination thereof.

10. The method of claim 1, wherein the at least one exosomal proteins are measured using a protein detection technique selected from liquid chromatography, mass spectrometry, trapped ion mobility spectrometry (TIMS), time-of-flight (TOF) mass spectrometry, enzyme linked immunoassay (ELISA), or combinations thereof.

11. The method of claim 1, wherein the subject diagnosed with the ovarian cancer is administered an anti-cancer therapy.

12. The method of claim 11, wherein the anti-cancer therapy comprises chemotherapy, immunotherapy, palliative therapy, surgery, or combinations thereof.

13. A method of treating or preventing a high grade serous ovarian carcinoma (HGSOC) in a subject, the method comprising:
a. isolating a serum sample from the subject,
b. measuring at least one exosomal protein selected from ANG-2, AGRIN, CD40, CPN1, CFH, CMET, CCNE1, Cyto P450 2E1, Cyclin E, CXCL1, CXCL3, FAS, G6-MP, GAL1, HGF, IL-6, IL-8, MAPK, MMP1, MMP3, NID1, PDL1, Pim-2 oncogene, PZP, SPP24, STAT3, pSTAT3, TETN, TETR, or VEGF1 in the serum sample,
c. detecting the HGSOC in the subject comprising increased levels of the at least one exosomal protein relative to a control subject, and
d. administering to the subject an anti-cancer therapy selected from chemotherapy, immunotherapy, radiation therapy, surgery, palliative care, or combinations thereof.

14. The method of claim 13, wherein the method measures at least 1 exosomal protein to about 30 exosomal proteins.

15. The method of claim 13, wherein the method distinguishes between an early stage HGSOC and an advanced stage HGSOC relative.

16. The method of claim 13, wherein the exosomal proteins are increased in a subject with the early stage HGSOC subject relative to the healthy control subject.

17. The method of claim 13, wherein the exosomal proteins are increased in a subject with the advanced stage HGSOC subject relative to the healthy control subject.

18. The method of claim 13, wherein the exosomal proteins are differentially expressed in the subject with the early stage HGSOC subject relative to the subject with the advanced stage HGSOC.

19. The method of claim 13, wherein the method further detects a protein not located in an exosome.

20. The method of claim 13, wherein the method further detects HE4, MUC16 (CA-125), or combinations thereof.

21. The method of claim 13, wherein the at least one exosomal proteins are measured using a protein detection technique selected from liquid chromatography, mass spectrometry, trapped ion mobility spectrometry (TIMS), time-of-flight (TOF) mass spectrometry, enzyme linked immunoassay (ELISA), or combinations thereof.

22.-40. (canceled)
